(19) United States
(12) Patent Application Publication
(10) Pub. No.: US 2022/0267271 A1
(43) Pub. Date: Aug. 25, 2022

(54) WNT ACTIVATORS AND METHODS OF USE

(71) Applicant: MAYO FOUNDATION FOR MEDICAL EDUCATION AND RESEARCH, Rochester, MN (US)

(72) Inventors: Guojun Bu, Jacksonville, FL (US); Thomas Caulfield, Jacksonville, FL (US); Yonghe Li, Ponte Vedra Beach, FL (US); Chia-Chen Liu, Jacksonville, FL (US)

(21) Appl. No.: 17/622,377

(22) PCT Filed: Jul. 8, 2020
(86) PCT No.: PCT/US20/41186

§ 371 Date: Dec. 23, 2021

Related U.S. Application Data
(60) Provisional application No. 62/871,301, filed on Jul. 8, 2019.

(51) Int. Cl. C07D 213/82; A61P 25/28; C07D 205/04; C07D 401/12; C07D 401/14

(52) U.S. Cl. CPC C07D 213/82; A61P 25/28; C07D 401/14; C07D 401/12; C07D 205/04

(57) ABSTRACT

Provided herein are compounds of Formula (I): and methods of activating Wnt using compounds as disclosed herein.

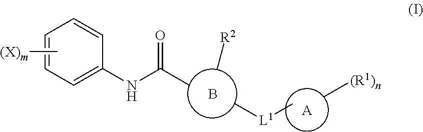

### BACKGROUND

Wnt/β-catenin signaling is involved in various differentiation events during embryonic development, and plays a critical role in adult tissue homeostasis and regeneration [1, 2]. Wnt proteins are secreted glycoproteins that bind to the extra-cellular cysteine-rich domain of the Frizzled (Fzd) receptor family and Wnt co-receptor the low density lipoprotein receptor-related protein 5 (LRP5) or LRP6 to activate the canonical β-catenin signaling pathway. The binding of Wnt to the Fzd/LRP5/6 receptor complex results in the inhibition of glycogen synthase kinase 3β (GSK3β) and the stabilization of cytosolic β-catenin. Stabilized β-catenin then translocates into the nucleus where it interacts with T-cell factor/lymphoid enhancing factor (TCF/LEF) to induce the expression of downstream targets [1, 2].

AD is the most common age-dependent neurodegenerative disorder characterized by progressive cognitive decline. It is well established that the aggregation and deposition of Aβ and hyperphosphorylated tau are associated with cognitive impairment and neuronal death in AD. As such, targeting Aβ production/clearance and inhibition of tau protein hyperphosphorylation are attractive strategies for AD therapy [3]. Moreover, neuronal loss is prominent in AD, and the promotion of neuronal survival and induction of neurogenesis are considered as promising treatment strategies of AD [4, 5]. Mounting evidence indicates that Wnt/β-catenin signaling is down-regulated in AD, and plays an important role in the pathogenesis of AD [6]. Particularly, we found the expression of Wnt co-receptor LRP6 is downregulated in AD brains [7], and deficiency in LRP6-mediated Wnt/β-catenin signaling contributes to synaptic abnormalities and an increase of amyloid pathology in AD [7]. In addition, two LRP6 SNPs and an alternative splice variant, which display impaired the signaling activity, are associated with increased risk of developing AD [8, 9]. Importantly, activation of Wnt/β-catenin signaling not only inhibits the Aβ production [10-12] and tau hyperphosphorylation [13-15], but also enhances synaptic plasticity [16, 17]. Moreover, Wnt/β-catenin signaling is the key positive regulator of neuronal survival, microglial survival and adult neurogenesis in the brain [14, 15, 18-24]. Therefore, activation of Wnt/β-catenin signaling represents an opportunity for rational design of targeted AD therapy (FIG. 1).

Studies in the past decade indicate that Wnt/β-catenin signaling plays a critical role in the regulation of bone mass and is involved in many disorders of bone [25, 26]. Osteoblast differentiation is the primary event of bone formation, and Wnt/β-catenin signaling increases bone formation via stimulation of the development of osteoblasts [25, 26]. Moreover, Wnt/β-catenin signaling is required to inhibit osteoclastic bone resorption by stimulating the expression of anti-osteoclastic factor OPG, the decoy receptor for RANKL, in osteoblast and osteocyte [25, 26]. Therefore, the Wnt/β-catenin signaling pathway is an attractive target for therapeutic intervention to restore bone strength in millions of patients with osteoporosis [25, 26]. Moreover, therapeutics with activation of Wnt/β-catenin signaling would also have applications in other diseases that are characterized by low bone mass and high bone fragility, such as osteogenesis imperfecta and bone fractures [25, 26].

Obesity is a risk factor for metabolic diseases and is often associated with co-morbidities such as diabetes [27, 28]. The Wnt/β-catenin signaling pathway is a regulator of adipogenesis. Activation of Wnt/β-catenin signaling leads to decreased levels of PPARγ and C/EBPα, the two transcription factors that activate the expression of adipocyte genes [29, 30]. Moreover, Wnt/β-catenin signaling is involved in lipid metabolism and glucose homeostasis, and mutations in LRP5 may lead to the development of diabetes and obesity [31]. Therefore, the Wnt/β-catenin signaling pathway is emerging as an attractive therapeutic target of obesity and type 2 diabetes [29, 31].

A need exists for Wnt modulators and methods of treating Wnt-related disorders.

### SUMMARY

Provided herein are compounds that can modulate Wnt. In particular, provided are compounds, or pharmaceutically acceptable salts thereof, having a structure of Formula (I) or (I′):

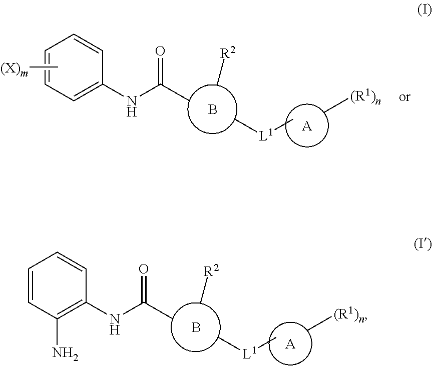

wherein ring B-R² is

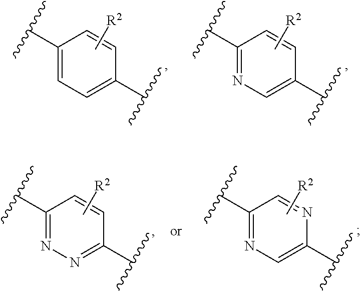

L¹ is NH—CO—C₀₋₃alkylene or CO—NH—C₀₋₄alkylene; ring A is a 4-12-membered monocyclic, bicyclic, bridged, or spiro heterocycle comprising a nitrogen ring atom; each R¹ is independently H, C₁₋₆alkyl, halo, C₁₋₆haloalkyl, C₁₋₃alkylene-O—C₁₋₃alkyl, C₀₋₃alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl; R² is H, F, OH, OMe, or NH₂; each X is independently NH₂, NMe₂, F, or CF₃, m is 1 or 2, and n is 1, 2, or 3, with the proviso that when ring A comprises piperidinyl, at least one R¹ is other than H. Also provided are compounds as listed in Table A, below, or a pharmaceutically acceptable salt thereof. In some cases, the compound or salt is listed in Table B, below.

Also provided are methods of activating Wnt in a cell comprising contacting the cell with a compound as disclosed herein, or a pharmaceutically acceptable salt thereof.

Further provided are methods of treating a neurological disorder in a subject in need thereof comprising administering to the subject a therapeutically effective amount of a compound as disclosed herein, or a pharmaceutically acceptable salt thereof. In various cases, the neurological disorder is selected from the group consisting of Alzheimers disease, frontotemporal dementias, dementia with lewy bodies, a prion disease, Parkinsons disease, Huntingtons disease, progressive supranuclear palsy, corticobasal degeneration, multiple system atrophy, amyotrophic lateral sclerosis (ALS), inclusion body myositis, autism, degenerative myopathy, diabetic neuropathy, endocrine neuropathy, orthostatic hypotension, multiple sclerosis and Charcot-Marie-Tooth disease. In some cases, the neurological disorder is Alzheimer's disease.

Further provided are methods of treating bone degeneration in a subject in need thereof comprising administering to the subject a therapeutically effective amount of a compound as disclosed herein, or a pharmaceutically acceptable salt thereof.

### BRIEF DESCRIPTION OF THE FIGURES

FIG. 1 shows the activation of WNT/β-catenin signaling as a promising therapeutic strategy for Alzheimer's Disease (AD).

FIG. 2 shows that CI-994 stimulates Wnt/β-catenin signaling by enhancing LRP6 expression. (A) structures of niciosamide and CI-994; (B) HEK293 cells were transfected with Super8XTOPFlash and β galactosidase vectors along with Wnt3A or control vector. After 24 hours incubation, cells were treated with CI-994 at the indicated concentration for 24 hr. The luciferase activity was then measured and normalized to the activity of the β-galactosidase. (C) HT1080 cells stably transduced LRP6 with HA tag were treated with niciosamide and CI-994 at the indicated concentrations for 24 h. The levels of HA-LRP6 were examined by Western blot with anti-HA antibody. (D) Western blots showing the depletion of LRP6 after 2 days of transiently transfection of 50 nM LRP6 siRNA in HEK293 cells. ON-TARGETplus double-stranded siRNA oligomers against human LRP6 and non-specific scrambled siRNA control (Stealth RNAi™ siRNA Negative Control, Med GC) were purchased from Thermo Scientific. siRNA LRP6-1, siRNA LRP6-2 and control siRNA were Thermo Scientific catalog numbers J-003845-09, J-003845-11 and 12935300, respectively. (E) HEK293 cells were transfected with LRP6 siRNAs, and after 8 h incubation were further transfected with Super8XTOPFlash construct and β galactosidase-expressing vector. After 24 h incubation, the cells were treated with CI-994 (16 μM) for 24 h. The luciferase activity was then measured and normalized to β-galactosidase activity. All the values are the average of triplicate determinations with the SD values indicated by error bars. **P<0.01 versus corresponding control value.

FIG. 3 shows that CI-994 activates Wnt/β-catenin signaling and suppresses BACE1 expression in N2a cells. (A,C) N2a cells were treated with CI-994 at the indicated concentration for 24 h, and the levels of β-catenin (A) and BACE1 (C) were examined by Western blotting. (B,D) The band intensity of β-catenin and BACE1 was quantified and normalized to the corresponding signal for actin. The results represented in the histograms are shown as the mean±SD and are the average of three independent experiments. *P<0.05, **P<0.01 versus corresponding control value.

FIG. 4 shows that 1015 and 1039 activate Wnt/β-catenin signaling in HEK293 cells. HEK293 cells were transfected with SuperXTOPFlash and β-galactosidase vectors along with Wnt3A or control vector. After 24 h incubation, cells were treated with 1015 or 1039 at the indicated concentrations for 24 h. The luciferase activity was then measured and normalized to the activity of the β-galactosidase. All the values are the average of triplicate determinations with the SD values indicated by error bars. This experiment is representative of at least three such experiments performed with similar data.

FIG. 5 shows compounds as disclosed herein activate Wnt/β-catenin signaling in N2a cells. N2a cells were treated with 1015, 1011, or 1020 at the indicated concentration for 24 h. (A) the levels of total cellular β-catenin were examined by Western blotting. (B) The intensity of the β-catenin bands were quantified and normalized to the corresponding signal for actin. The results represented in the histograms are shown as the mean±SD and are the average of 3-4 independent experiments, and the EC₅₀ values were calculated with graphpad prism.

FIG. 6 shows that Wnt activators suppress BACE1 expression in N2a cells. N2a cells were treated with 1015 and 1020 at the indicated concentrations for 24 h. (A) the levels of BACE1 expression were examined by Western blotting. (B) The intensity of BACE1 bands were quantified and normalized to the corresponding signal for actin. The results represented in the histograms are shown as the mean±SD and are the average of 3-4 independent experiments. *P<0.05, **P<0.01 versus corresponding control value.

FIG. 7 shows that 1011 inhibits GSK3β activity in N2a cells. N2a cells were treated with 1011 at the indicated concentrations for 24 h. (A) the levels of β-catenin, GSK3β and GSK3β phosphorylation (Ser9) were examined by Western blotting. (B) the intensity of the GSK3β phosphorylation bands were quantified and normalized to the corresponding signal for total GSK3β. The results represented in the histograms are shown as the mean±SD and are the average of 3 independent experiments. *P<0.05, **P<0.01 versus corresponding control value.

FIG. 8 shows that 1011 inhibits tau phosphorylation in N2a cells. N2a cells were treated with 1011 at the indicated concentrations for 24 h. (A) the levels of β-catenin, tau and tau phosphorylation were examined by Western blotting. (B) the intensity of the tau phosphorylation bands were quantified and normalized to the corresponding signal for total tau. The results represented in the histograms are shown as the mean±SD and are the average of 3 independent experiments. *P<0.05, **P<0.01 versus corresponding control value.

FIG. 9 shows that Wnt activators induce osteoblastic differentiation in C2C12 cells. C2C12 cells were seeded into 24-well plates (1×10⁵ cells/well). After culture overnight, the cells were treated with a Wnt activator at 0.8 μM (A) and 1.6 μM (B) for 72 h. The ALP activity was measured by ALP assay kit from AnaSpec (AS-72146). All the values are the average of triple determinations with the s.d. indicated by error bars. *P<0.05, **P<0.01 versus corresponding control value. This experiment is a representation of two such experiments performed with similar data.

FIG. 10 shows that osteoblastic differentiation induced by CI-994 and other Wnt activators (1009, 1011, and 1015) in C2C12 cells. C2C12 cells were seeded into 24-well plates (1×10⁵ cells/well). After culture overnight, the cells were treated with the noted compounds at 0, 0.1, 0.2, 0.4, 0.8, 1.6, 3.2, 6.4 and 12.8 μM for 72 hours. The ALP activity was measured by ALP assay kit from AnaSpec (AS-72146). All the values are the average of triple determinations with the s.d. indicated by error bars.

FIG. 11 shows that Wnt activators activate Wnt/β-catenin signaling by enhancing LRP6 expression. (A) HT1080 cells stably transduced with human LRP6 with HA tag were treated with niciosamide (1 μM) and other compounds as noted (1 μM) for 24 hr. The levels of HA-LRP6 were examined by Western blots with anti-HA antibody. All samples were also probed with anti-actin antibody to verify equal loading. This experiment is a representative of two such experiments performed with similar data. (B) N2a cells were treated with 1015 and 1011 at the indicated concentrations for 24 h. The expression levels of LRP6, β-catenin and actin were examined by Western blotting. This experiment is a representative of three such experiments performed with similar data. (C) HEK293 cells were transfected with LRP6 siRNAs, and after 8 h incubation were further transfected with Super8XTOPFlash construct and β galactosidase-expressing vector. ON-TARGETplus double-stranded siRNA oligomers against human LRP6 and non-specific scrambled siRNA control (Stealth RNAi™ siRNA Negative Control, Med GC) were purchased from Thermo Scientific. siRNA LRP6-1, siRNA LRP6-2 and control siRNA were Thermo Scientific catalog numbers J-003845-09, J-003845-11 and 12935300, respectively. After 24 h incubation, the cells were treated with 1011, 1015 or 1023 at 1 μM for 24 h. The luciferase activity was then measured and normalized to β-galactosidase activity. All the values are the average of triplicate determinations with the SD values indicated by error bars. **P<0.01 versus corresponding control value. This experiment is a representative of two such experiments performed with similar data.

FIG. 12 shows the effects of various compounds on HDAC activity. HDAC enzymatic activities of compounds were measured by fluorometric HDAC Activity Assay kit (Abcam. #ab156064). Trichostatin A, a known HDAC inhibitor, was used as the positive control. All the values are the average of triple determination with the s.d. indicated by error bars. **P<0.01 versus the corresponding control value.

FIG. 13 shows the cytotoxicity of 1011 and 1015. The human brain endothelial capillary hCMEC/D3 cells were cultured with compounds at the indicated concentrations for 24 h. Cell viability was assessed by the Cell TitreGlo Assay system from Promega. Data represent mean±SD from three independent experiments. *P<0.05.

FIG. 14 shows the metabolic stability of compounds in mouse liver microsomes. Metabolic stability of several compounds (1009, 1011, 1015, 1020, and 1056) and 2 reference compounds (propranolol and imipramine) were determined in mouse liver microsomes at five time points over 40 min using HPLC-MS. Metabolic stability is defined as the percentage of parent compound lost over time in the presence of a metabolically active test system.

FIG. 15 shows the in vivo pharmacokinetic studies of 1015. (A) Plasma and brain concentration—time curves of 1015 in male Balb/c mice following intravenous (2 mg/kg) administration (n=4 at each time point). (B) in vivo pharmacokinetic parameters of 1015. B/P: brain/plasma concentration ratio (30 min after IV injection with 2 mg/kg) calculated using 1 g/mL brain density.

FIG. 16 shows that 1015 activates Wnt/β-catenin signaling in human iPSC-derived neurons, where (A) shows human iPSC-derived neurons were treated with 1015 at the indicated concentrations for 24 h, the level of LRP6 expression was examined by Western blotting. Human iPSC-derived neurons were treated with 1015 (0.4 μM) for 48 h, and the mRNA levels of axin2 (B), neuroD1 (C) and SOX2 (D) were determined by qPCR. All the values are the average of three independent experiments with the SE values indicated by error bars. * P<0.05, ** P<0.01 verse corresponding control. (E) Human iPSC-derived neurons were treated with 1015 (0.4 μM) for 48 h, and the levels of phopspho-tau (p-tau) and total tau were determined by Western blotting with specific antibodies AT8 and HT7, respectively. This experiment is a representative of two such experiments performed with similar data.

FIG. 17 shows 1015 prevents body weight loss and increases cognitive function in PS19 mice. PS19 mice at 33 weeks of age were treated with 1015 (IP, 15 mg/kg) or vehicle [Kolliphor HS-physiological saline (20%:80%)] by a once daily dose schedule for 8 weeks (5 injections per week). (A) Body weight changes during the course of treatment were assessed. (B) The level of Wnt specific target neuroD1 in cortex was determined by qPCR. All the values are the average of from 5-7 mice with the SE values indicated by error bars.* P<0.05, * P<0.01 verse Non-Tg treated with vehicle or PS19 mice treated with vehicle.

FIG. 18 shows 1015 suppresses tau phosphorylation in cortex of PS19 mice. (A) PS19 mice at 33 weeks of age were treated with 1015 (IP, 15 mg/kg) or vehicle [Kolliphor HS-physiological saline (20%:80%)] by a once daily dose schedule for 8 weeks (5 injections per week). The levels of tau phosphorylation (p-tau) and total tau in cortex were determined by Western blotting with p-tau antibody AT8, p-tau antibody AT180, and tau antibody HT7, respectively. (B) The intensities of p-tau bands were quantified and normalized to the corresponding signal for actin. The results represented in the histograms are shown as the mean±SE, n=5.

FIG. 19 shows 1015 suppresses neuroinflammation in PS19 mice. PS19 mice at 33 weeks of age were treated with 1015 (IP, 15 mg/kg) or vehicle [Kolliphor HS-physiological saline (20%:80%)] by a once daily dose schedule for 8 weeks (5 injections per week). (A-D) The levels of cytokines IL6, TNFa, TGF1b and IL1b in cortex were determined by qPCR. All the values are the average of from 5-7 mice with the SE values indicated by error bars. * P<0.05, * P<0.01, ** P<0.001.

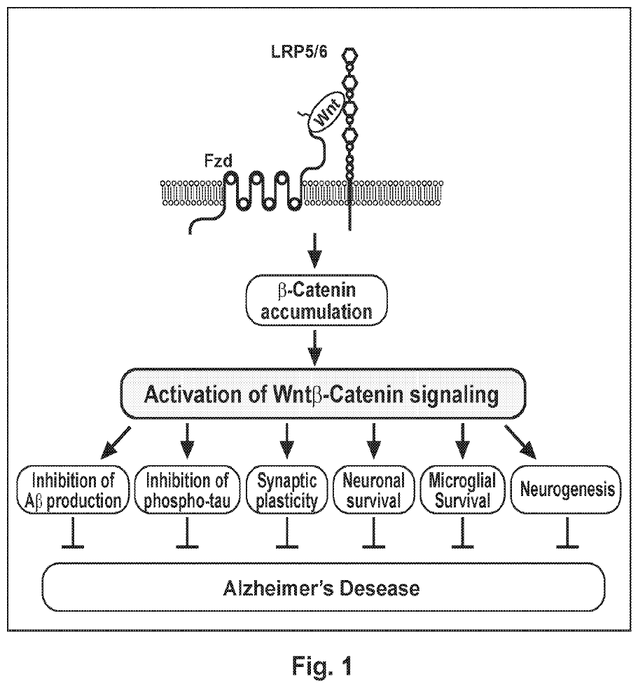

### DETAILED DESCRIPTION

Provided herein are compounds of formula (I), or more specifically (I′), or pharmaceutically acceptable salts thereof:

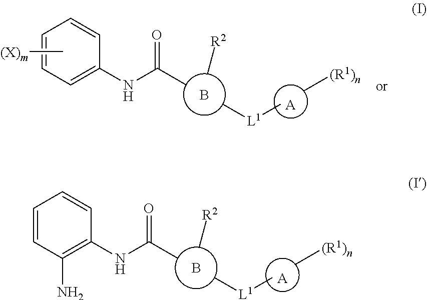

wherein

ring B-R² is

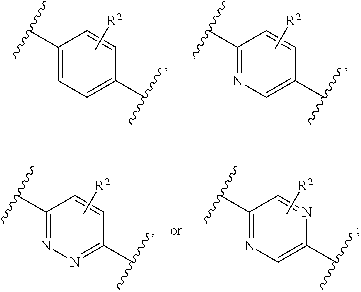

L¹ is NH—CO—C₀₋₃alkylene or CO—NH—C₀₋₄alkylene;
ring A is a 4-12-membered monocyclic, bicyclic, bridged, or spiro heterocycle comprising a nitrogen ring atom;
each R¹ is independently H, C₁₋₆alkyl, halo, C₁₋₆haloalkyl, C₁₋₃alkylene-O—C₁₋₃alkyl, C₀₋₃alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl;
R² is H, F, OH, OMe, or NH₂;
each X is independently NH₂, NMe₂, F, or CF₃;
n is 1, 2, or 3; and
m is 1 or 2,
   with the proviso that when ring A comprises piperidinyl, at least one R¹ is other than H.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. A “haloalkyl” group is an alkyl group having at least one halo substituent. In some cases, the haloalkyl comprises 1, 2, or 3 halo substituents, or can comprise a perhaloalkyl (i.e., all hydrogen atoms of the alkyl group are substituted with a halo). Non-limiting examples of haloalkyl include trifluoromethyl, fluoroethyl, difluoroethyl, and trifluoroethyl.

The term “alkylene” used herein refers to an alkyl group having a substituent. For example, an alkylene group can be —CH₂CH₂— or —CH₂— or —CH₂CH(CH₃)—. The term Cₙ means the alkylene group has “n” carbon atoms. For example, C₁₋₄alkylene refers to an alkylene group having a number of carbon atoms encompassing the entire range, as well as all subgroups, as previously described for “alkyl” groups. A C₀alkylene group refers to a direct bond. Unless otherwise indicated, an alkylene group can be an unsubstituted alkylene group or a substituted alkylene group.

As used herein, the term “carbocycle” refers to an aliphatic cyclic hydrocarbon group containing three to eight carbon atoms (e.g., 3, 4, 5, 6, 7, or 8 carbon atoms). The term Cₙ means the carbocycle group has “n” carbon atoms. For example, C₅ carbocycle refers to a carbocycle group that has 5 carbon atoms in the ring. C₆-C₈ carbocycle refers to carbocycle groups having a number of carbon atoms encompassing the entire range (e.g., 6 to 8 carbon atoms), as well as all subgroups (e.g., 6-7, 7-8, 6, 7, and 8 carbon atoms). Nonlimiting examples of carbocycle groups include cyclopropyl, cyclobutyl, cyclopentyl, cyclohexyl, cycloheptyl, and cyclooctyl. Unless otherwise indicated, a carbocycle group can be an unsubstituted carbocycle group or a substituted carbocycle group.

As used herein, the term “heterocycle” is defined similarly as carbocycle, except the ring contains one to three heteroatoms independently selected from oxygen, nitrogen, and sulfur. In particular, the term “heterocycle” refers to a ring containing a total of three to twelve atoms (e.g., 3-8, 5-8, 3-6, 4-12, 3, 4, 5, 6, 7, 8, 9, 10, 11, or 12), of which 1, 2, or 3 of the ring atoms are heteroatoms independently selected from the group consisting of oxygen, nitrogen, and sulfur, and the remaining atoms in the ring are carbon atoms. The heterocycle can be monocyclic, bicyclic, bridged, or spiro heterocycle. In some cases, the heterocycle can be a 4-12 membered ring and comprises only one ring heteroatom—and in particular embodiments, a sole nitrogen ring heteroatom. Nonlimiting examples of heterocycle groups include piperdine, pyrazolidine, tetrahydrofuran, tetrahydropyran, dihydrofuran, morpholine, and the like. In some cases, the heterocycle can be azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl.

Carbocycle and heterocycle groups can be saturated or partially unsaturated ring systems optionally substituted with, for example, an R¹ group as disclosed herein. Heterocycle groups optionally can be further N-substituted with an R¹ group, e.g., alkyl (for example, methyl or ethyl), alkylene-carbocycle, alkylene-aryl, and alkylene-heteroaryl.

As used herein, the term “aryl” refers to a monocyclic or bicyclic aromatic group, having 6 to 10 ring atoms. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Aryl groups can be isolated (e.g., phenyl) or fused to another aryl group (e.g., naphthyl), a carbocycle group (e.g. tetrahydronaphthyl), a heterocycloalkyl group, and/or a heteroaryl group.

As used herein, the term “heteroaryl” refers to a monocyclic or bicyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl is a 5-7 membered monocyclic ring having 1 to four ring heteroatoms. In some cases, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

In various cases, m is 1. In some cases, m is 1 and X is NH₂; and in some specific cases, X is meta to the amide bond on the phenyl ring, and in other specific cases, X is para to the amide bond on the phenyl ring. In some cases, m is 1 and X is NMe₂, and in some specific cases, X is ortho to the amide bond on the phenyl ring. In some cases, m is 1 and X is CF₃, and in some specific cases, X is meta to the amide bond on the phenyl ring. In some cases, m is 1, and X is F, and in some specific cases, X is ortho to the amide bond on the phenyl ring. In some cases, m is 2. In various cases, m is 2 and one X is NH₂ and one X is F, and in some specific cases, each X is ortho to the amide bond on the phenyl ring.

In various cases, ring A of Formula (I) or (I′) can comprise azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl. In some cases, the substituent on ring A, R¹, can be fluoro, cyclopropyl, cyclopentyl, cyclohexyl, cycloheptyl, CH₂cyclohexyl, CH₂cyclopenyl, methyl, CH₂CH₂OCH₃, isopropyl, difluoroethyl, trifluoroethyl, tetrahydropyranyl, CH₂-(methyl-isoxazolyl), methyl-pyrazolyl, CH₂CH₂phenyl, CH₂phenyl, CH₂(methoxyphenyl), or phenyl.

In various cases, ring B-R² is

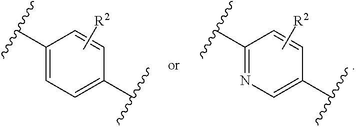

in various cases, ring B-R² is

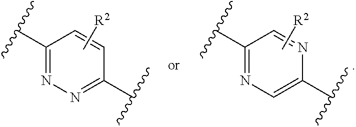

In some cases, R² is H. In some cases, R² is F. In some cases, R² is OH, OMe, or NH₂.

In various cases, L¹ is NHCO—C₀₋₄alkylene. In various cases, L¹ is CONH—C₀₋₄alkylene. In some cases, L¹ is CONHCH₂, or CONHCH₂CH₂, or CONHCH₂CH(CH₃).

In various cases, the compound can have a structure of Formula (IA), (IA′), (IA″), (IA″′), (IB), (IB′), (IC), or (IC′):

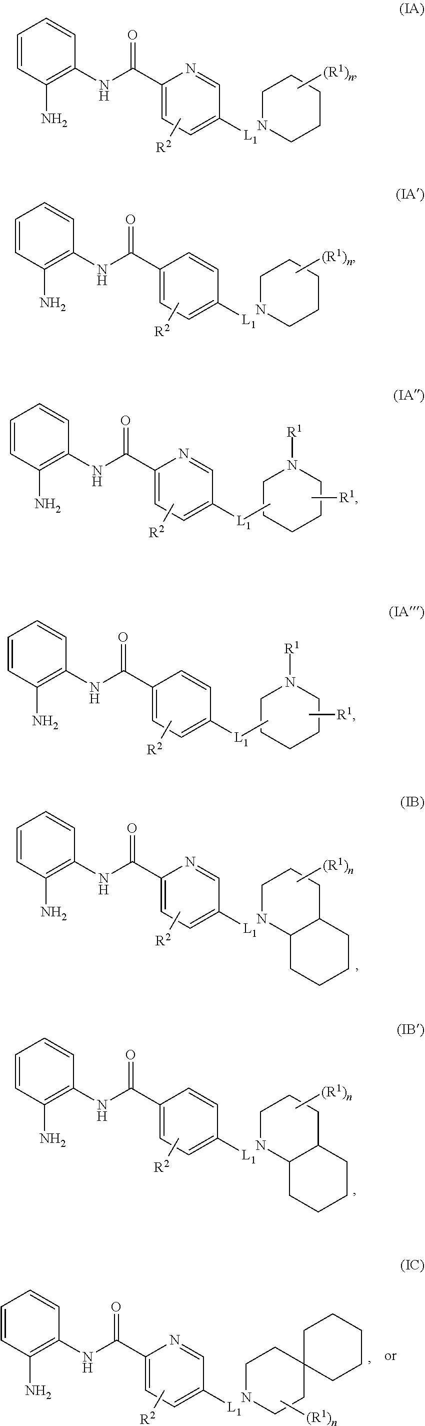

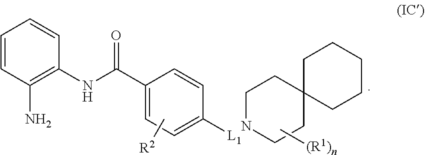

In some cases, the compound has a structure of Formula (ID) or (ID′):

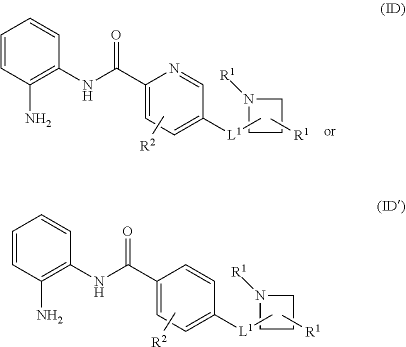

wherein when L¹ is attached to the ring nitrogen, R¹ on the ring nitrogen is null. In various cases, the compound has a structure of Formula (IE) or (IE′):

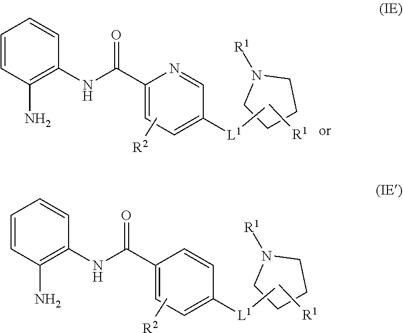

wherein when L¹ is attached to the ring nitrogen, R¹ on the ring nitrogen is null.

Specific compounds contemplated in the present disclosure include those as provided in Table A, below, or a pharmaceutically acceptable salt thereof.

TABLE A

| STRUCTURE | COMPOUND |
|---|---|
| 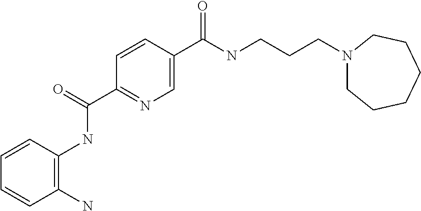 [SMILES: Nc1ccccc1NC(=O)c1ccc(C(=O)NCCCN2CCCCCC2)cn1] | 1001 |
| 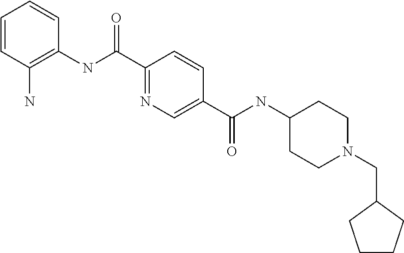 [SMILES: Nc1ccccc1NC(=O)c1ccc(C(=O)NC2CCN(CC3CCCC3)CC2)cn1] | 1002 |
| 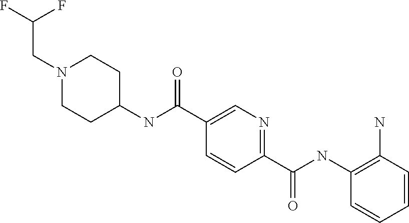 [SMILES: Nc1ccccc1NC(=O)c1ccc(C(=O)NC2CCN(CC(F)F)CC2)cn1] | 1003 |
| 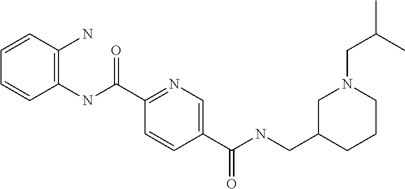 [SMILES: CC(C)CN1CCCC(CNC(=O)c2ccc(C(=O)Nc3ccccc3N)nc2)C1] | 1004 |
| 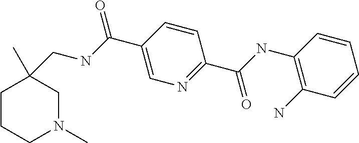 [SMILES: CN1CCCC(C)(CNC(=O)c2ccc(C(=O)Nc3ccccc3N)nc2)C1] | 1005 |
| 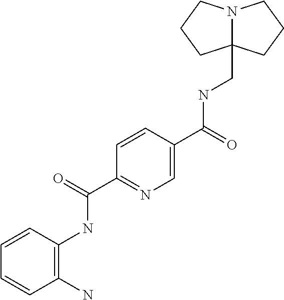 [SMILES: Nc1ccccc1NC(=O)c1ccc(C(=O)NCC23CCCN2CCC3)cn1] | 1006 |
| 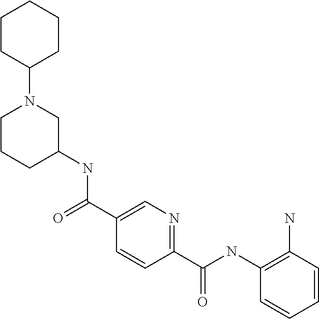 [SMILES: Nc1ccccc1NC(=O)c1ccc(C(=O)NC2CCCN(C3CCCCC3)C2)cn1] | 1007 |
| 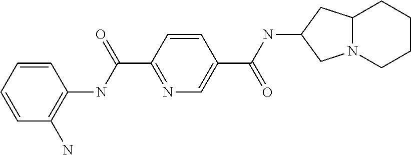 [SMILES: Nc1ccccc1NC(=O)c1ccc(C(=O)NC2CC3CCCCN3C2)cn1] | 1008 |
| 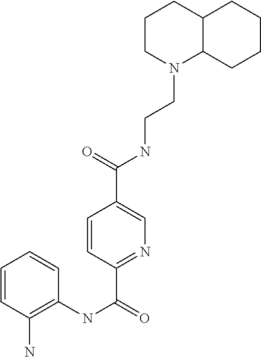 [SMILES: Nc1ccccc1NC(=O)c1ccc(C(=O)NCCN2CCCC3CCCCC32)cn1] | 1009 |
| 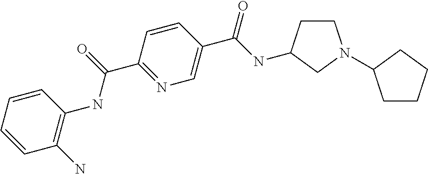 [SMILES: Nc1ccccc1NC(=O)c1ccc(C(=O)NC2CCN(C3CCCC3)C2)cn1] | 1010 |
| 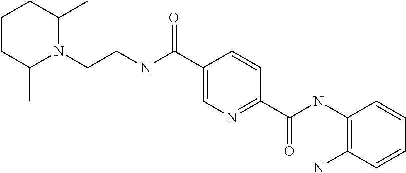 [SMILES: CC1CCCC(C)N1CCNC(=O)c1ccc(C(=O)Nc2ccccc2N)nc1] | 1011 |
| 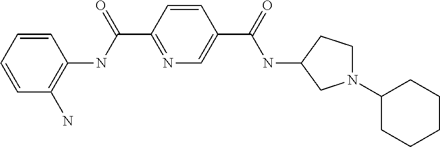 [SMILES: Nc1ccccc1NC(=O)c1ccc(C(=O)NC2CCN(C3CCCCC3)C2)cn1] | 1012 |
| 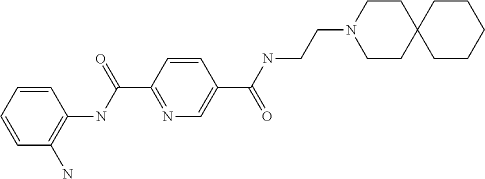 [SMILES: Nc1ccccc1NC(=O)c1ccc(C(=O)NCCN2CCC3(CCCCC3)CC2)cn1] | 1013 |
| 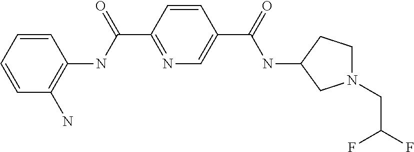 [SMILES: Nc1ccccc1NC(=O)c1ccc(C(=O)NC2CCN(CC(F)F)C2)cn1] | 1014 |
| 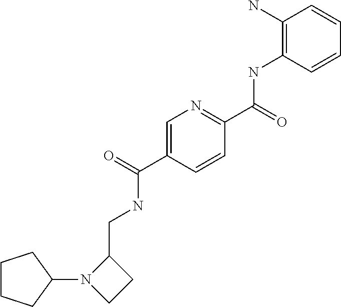 [SMILES: Nc1ccccc1NC(=O)c1ccc(C(=O)NCC2CCN2C2CCCC2)cn1] | 1015 |
| 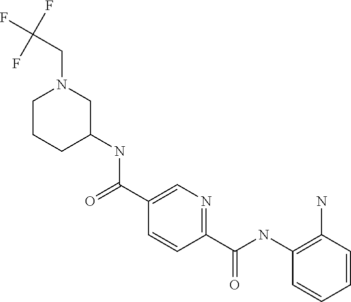 [SMILES: Nc1ccccc1NC(=O)c1ccc(C(=O)NC2CCCN(CC(F)(F)F)C2)cn1] | 1016 |
| 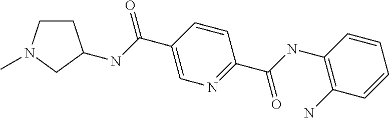 [SMILES: CN1CCC(NC(=O)c2ccc(C(=O)Nc3ccccc3N)nc2)C1] | 1017 |
| 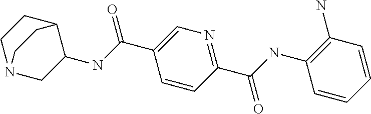 [SMILES: Nc1ccccc1NC(=O)c1ccc(C(=O)NC2CN3CCC2CC3)cn1] | 1018 |
| 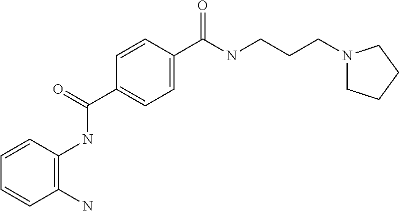 [SMILES: Nc1ccccc1NC(=O)c1ccc(C(=O)NCCCN2CCCC2)cc1] | 1019 |
| 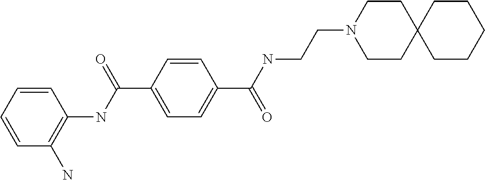 [SMILES: Nc1ccccc1NC(=O)c1ccc(C(=O)NCCN2CCC3(CCCCC3)CC2)cc1] | 1020 |
| 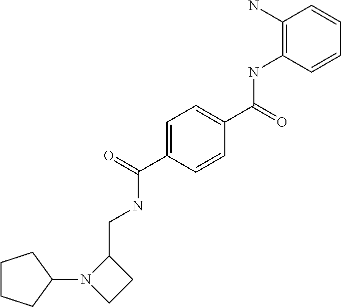 [SMILES: Nc1ccccc1NC(=O)c1ccc(C(=O)NCC2CCN2C2CCCC2)cc1] | 1021 |
| 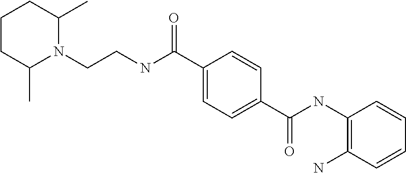 [SMILES: CC1CCCC(C)N1CCNC(=O)c1ccc(C(=O)Nc2ccccc2N)cc1] | 1022 |
| 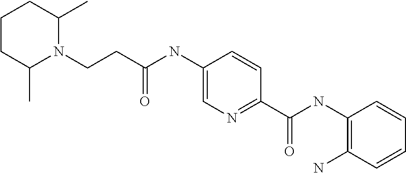 [SMILES: CC1CCCC(C)N1CCC(=O)Nc1ccc(C(=O)Nc2ccccc2N)nc1] | 1023 |
| 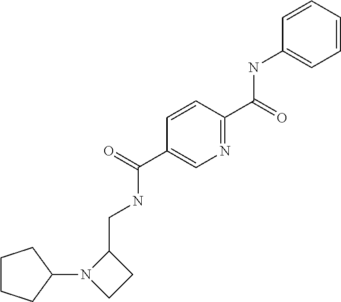 [SMILES: O=C(NCC1CCN1C1CCCC1)c1ccc(C(=O)Nc2ccccc2)nc1] | 1024 |
| 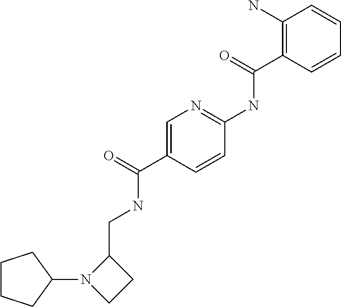 [SMILES: Nc1ccccc1C(=O)Nc1ccc(C(=O)NCC2CCN2C2CCCC2)cn1] | 1025 |
| 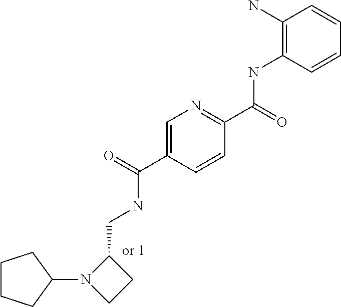 [SMILES: Nc1ccccc1NC(=O)c1ccc(C(=O)NC[C@@H]2CCN2C2CCCC2)cn1] | 1026 |
| 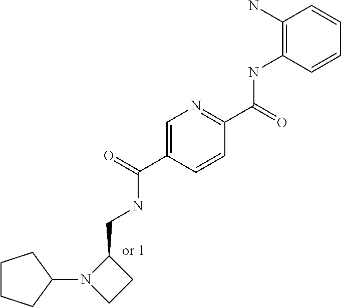 [SMILES: Nc1ccccc1NC(=O)c1ccc(C(=O)NC[C@H]2CCN2C2CCCC2)cn1] | 1027 |
| 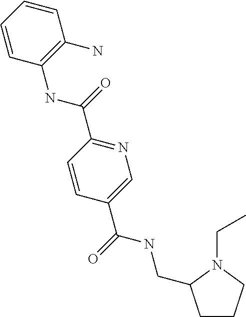 [SMILES: CCN1CCCC1CNC(=O)c1ccc(C(=O)Nc2ccccc2N)nc1] | 1028 |
| 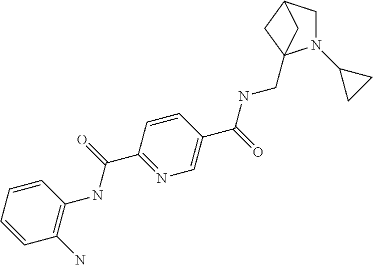 [SMILES: Nc1ccccc1NC(=O)c1ccc(C(=O)NCC23CC(CN2C2CC2)C3)cn1] | 1029 |
| 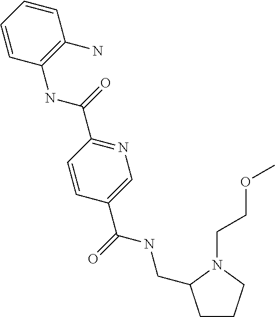 [SMILES: COCCN1CCCC1CNC(=O)c1ccc(C(=O)Nc2ccccc2N)nc1] | 1030 |
| 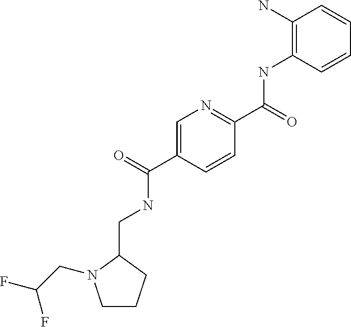 [SMILES: Nc1ccccc1NC(=O)c1ccc(C(=O)NCC2CCCN2CC(F)F)cn1] | 1031 |
| 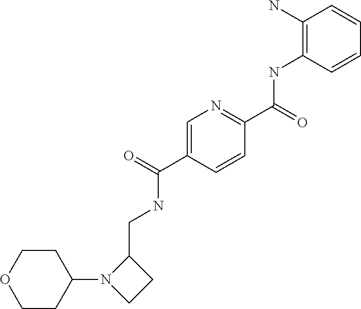 [SMILES: Nc1ccccc1NC(=O)c1ccc(C(=O)NCC2CCN2C2CCOCC2)cn1] | 1032 |
| 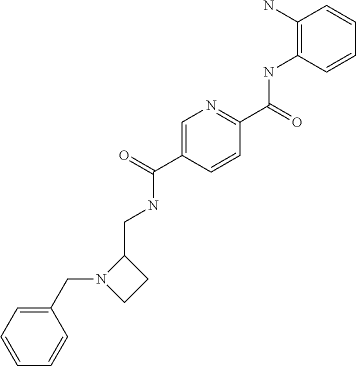 [SMILES: Nc1ccccc1NC(=O)c1ccc(C(=O)NCC2CCN2Cc2ccccc2)cn1] | 1033 |
| 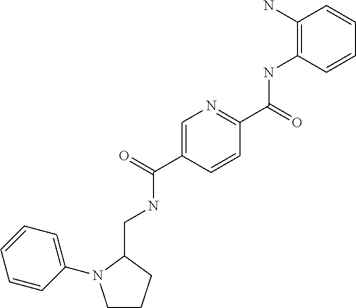 [SMILES: Nc1ccccc1NC(=O)c1ccc(C(=O)NCC2CCCN2c2ccccc2)cn1] | 1034 |
| 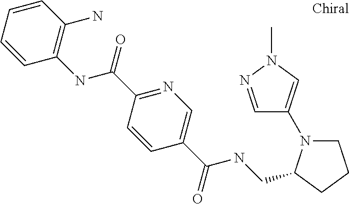 [SMILES: Cn1cc(N2CCC[C@@H]2CNC(=O)c2ccc(C(=O)Nc3ccccc3N)nc2)cn1] | 1035 |
| 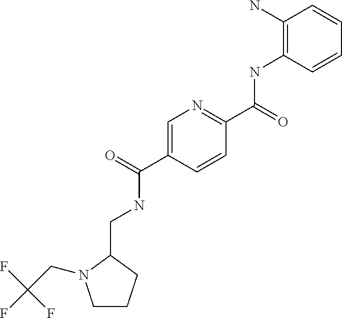 [SMILES: Nc1ccccc1NC(=O)c1ccc(C(=O)NCC2CCCN2CC(F)(F)F)cn1] | 1036 |
| 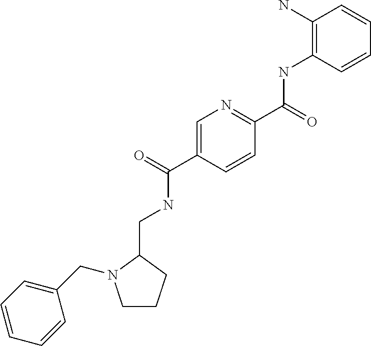 [SMILES: Nc1ccccc1NC(=O)c1ccc(C(=O)NCC2CCCN2Cc2ccccc2)cn1] | 1037 |
| 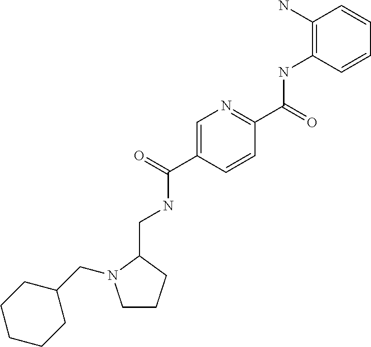 [SMILES: Nc1ccccc1NC(=O)c1ccc(C(=O)NCC2CCCN2CC2CCCCC2)cn1] | 1038 |
| 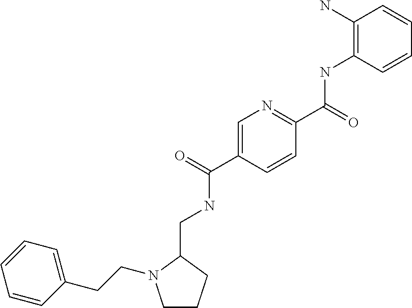 [SMILES: Nc1ccccc1NC(=O)c1ccc(C(=O)NCC2CCCN2CCc2ccccc2)cn1] | 1039 |
| 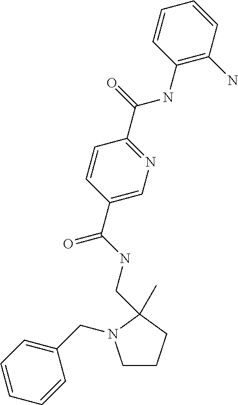 [SMILES: CC1(CNC(=O)c2ccc(C(=O)Nc3ccccc3N)nc2)CCCN1Cc1ccccc1] | 1040 |
| 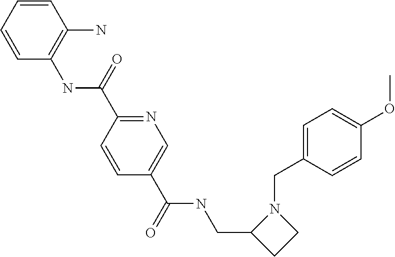 [SMILES: COc1ccc(CN2CCC2CNC(=O)c2ccc(C(=O)Nc3ccccc3N)nc2)cc1] | 1041 |
| 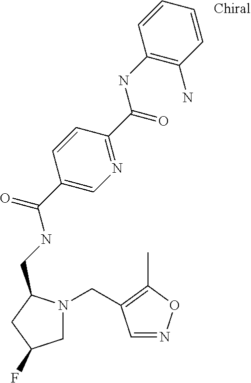 [SMILES: Cc1oncc1CN1C[C@@H](F)C[C@H]1CNC(=O)c1ccc(C(=O)Nc2ccccc2N)nc1] | 1042 |
| 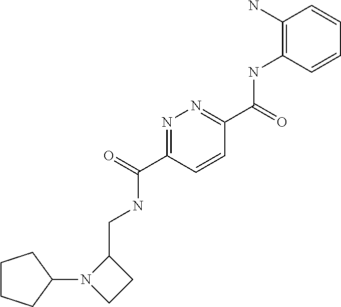 [SMILES: Nc1ccccc1NC(=O)c1ccc(C(=O)NCC2CCN2C2CCCC2)nn1] | 1043 |
| 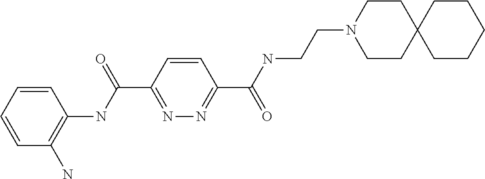 [SMILES: Nc1ccccc1NC(=O)c1ccc(C(=O)NCCN2CCC3(CCCCC3)CC2)nn1] | 1044 |
| 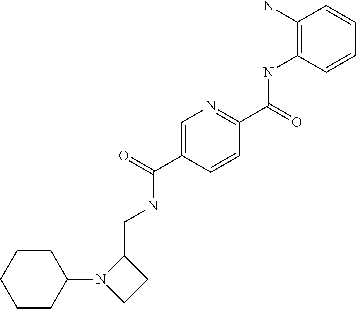 [SMILES: Nc1ccccc1NC(=O)c1ccc(C(=O)NCC2CCN2C2CCCCC2)cn1] | 1045 |
| 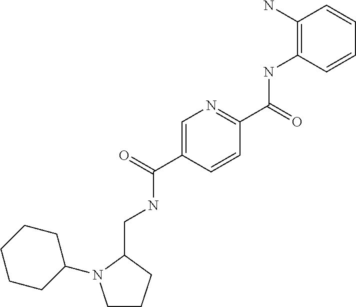 [SMILES: Nc1ccccc1NC(=O)c1ccc(C(=O)NCC2CCCN2C2CCCCC2)cn1] | 1046 |
| 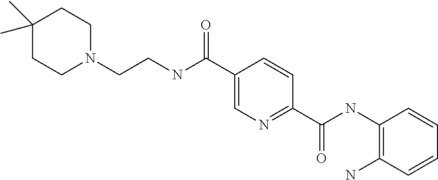 [SMILES: CC1(C)CCN(CCNC(=O)c2ccc(C(=O)Nc3ccccc3N)nc2)CC1] | 1047 |
| 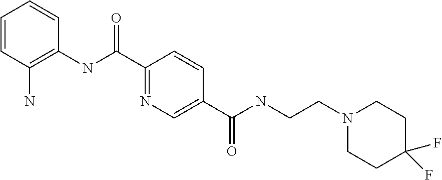 [SMILES: Nc1ccccc1NC(=O)c1ccc(C(=O)NCCN2CCC(F)(F)CC2)cn1] | 1048 |
| 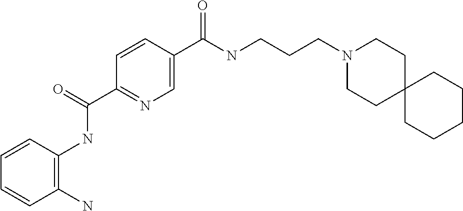 [SMILES: Nc1ccccc1NC(=O)c1ccc(C(=O)NCCCN2CCC3(CCCCC3)CC2)cn1] | 1049 |
| 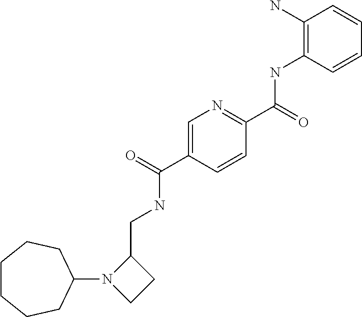 [SMILES: Nc1ccccc1NC(=O)c1ccc(C(=O)NCC2CCN2C2CCCCCC2)cn1] | 1050 |
| 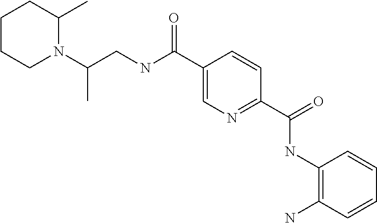 [SMILES: CC1CCCCN1C(C)CNC(=O)c1ccc(C(=O)Nc2ccccc2N)nc1] | 1051 |
| 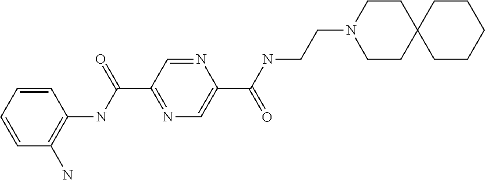 [SMILES: Nc1ccccc1NC(=O)c1cnc(C(=O)NCCN2CCC3(CCCCC3)CC2)cn1] | 1052 |
| 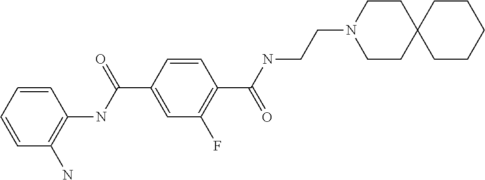 [SMILES: Nc1ccccc1NC(=O)c1ccc(C(=O)NCCN2CCC3(CCCCC3)CC2)c(F)c1] | 1053 |
| 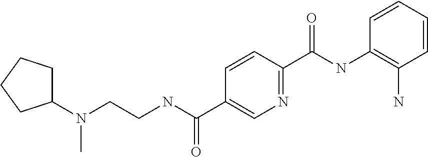 [SMILES: CN(CCNC(=O)c1ccc(C(=O)Nc2ccccc2N)nc1)C1CCCC1] | 1054 |
| 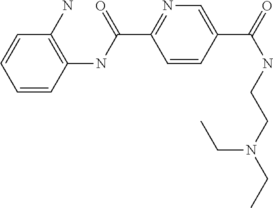 [SMILES: CCN(CC)CCNC(=O)c1ccc(C(=O)Nc2ccccc2N)nc1] | 1055 |
| 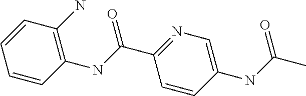 [SMILES: CC(=O)Nc1ccc(C(=O)Nc2ccccc2N)nc1] | 1056 |
| 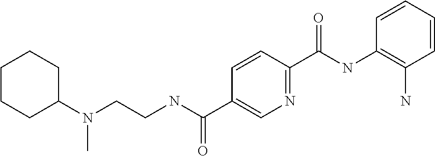 [SMILES: CN(CCNC(=O)c1ccc(C(=O)Nc2ccccc2N)nc1)C1CCCCC1] | 1057 |
| 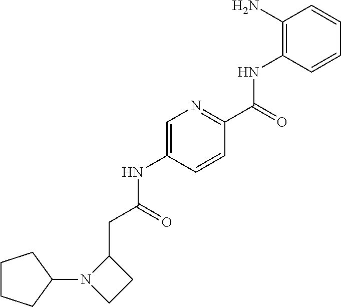 [SMILES: Nc1ccccc1NC(=O)c1ccc(NC(=O)CC2CCN2C2CCCC2)cn1] | 1058 |
| 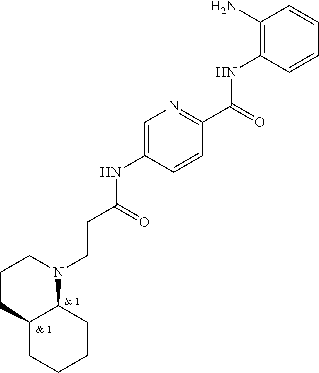 [SMILES: Nc1ccccc1NC(=O)c1ccc(NC(=O)CCN2CCC[C@@H]3CCCC[C@@H]32)cn1] | 1059 |
| 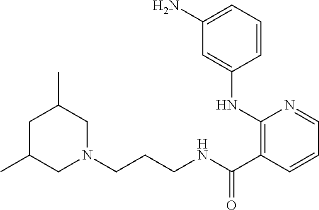 [SMILES: CC1CC(C)CN(CCCNC(=O)c2cccnc2Nc2cccc(N)c2)C1] | 1060 |
| 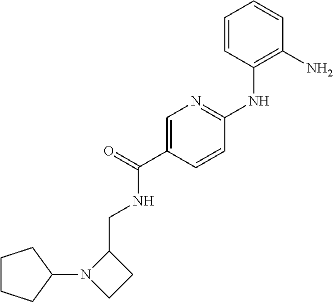 [SMILES: Nc1ccccc1Nc1ccc(C(=O)NCC2CCN2C2CCCC2)cn1] | 1061 |
| 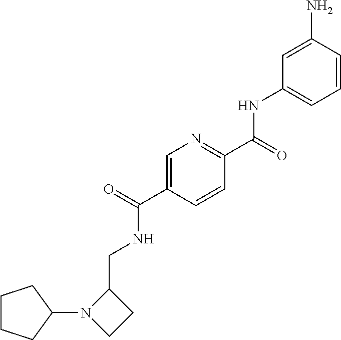 [SMILES: Nc1cccc(NC(=O)c2ccc(C(=O)NCC3CCN3C3CCCC3)cn2)c1] | 1062 |
| 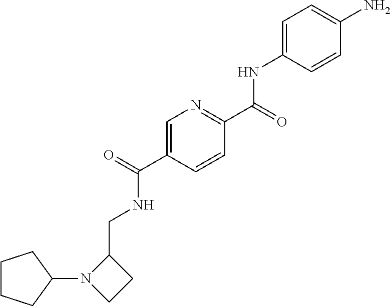 [SMILES: Nc1ccc(NC(=O)c2ccc(C(=O)NCC3CCN3C3CCCC3)cn2)cc1] | 1063 |
| 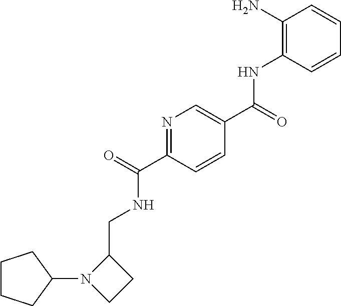 [SMILES: Nc1ccccc1NC(=O)c1ccc(C(=O)NCC2CCN2C2CCCC2)nc1] | 1064 |
| 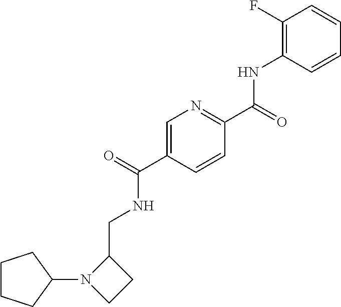 [SMILES: O=C(NCC1CCN1C1CCCC1)c1ccc(C(=O)Nc2ccccc2F)nc1] | 1065 |
| 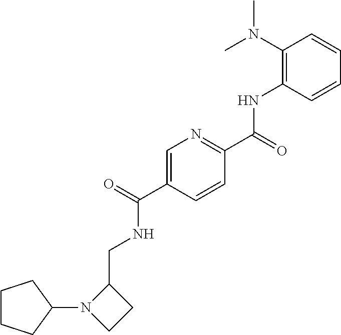 [SMILES: CN(C)c1ccccc1NC(=O)c1ccc(C(=O)NCC2CCN2C2CCCC2)cn1] | 1066 |
| 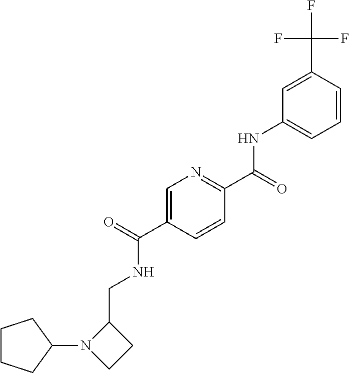 [SMILES: O=C(NCC1CCN1C1CCCC1)c1ccc(C(=O)Nc2cccc(C(F)(F)F)c2)nc1] | 1067 |
| 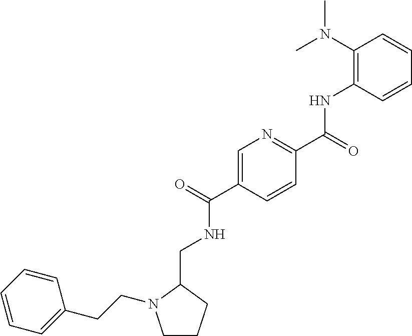 [SMILES: CN(C)c1ccccc1NC(=O)c1ccc(C(=O)NCC2CCCN2CCc2ccccc2)cn1] | 1068 |
| 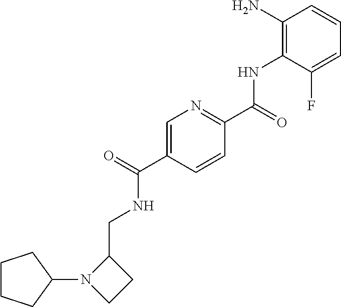 [SMILES: Nc1cccc(F)c1NC(=O)c1ccc(C(=O)NCC2CCN2C2CCCC2)cn1] | 1069 |
| 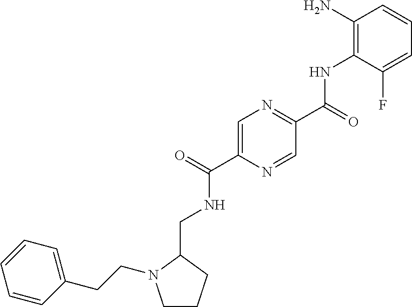 [SMILES: Nc1cccc(F)c1NC(=O)c1cnc(C(=O)NCC2CCCN2CCc2ccccc2)cn1] | 1070 |

In some cases, the compound of Table A is 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1048, 1047, 1048, 1049, 1050, 1051, 1052, 1053, 1054, 1055, 1056, or 1057.

In some cases, a compound of the present disclosure is as shown in Table B, or a pharmaceutically acceptable salt thereof.

TABLE B

| COMPOUND | ID |
|---|---|
| 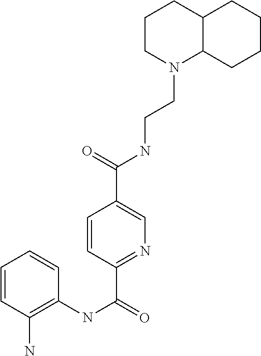 [SMILES: Nc1ccccc1NC(=O)c1ccc(C(=O)NCCN2CCCC3CCCCC32)cn1] | 1009 |
| 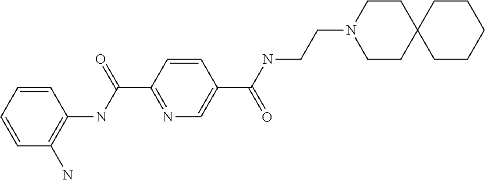 [SMILES: Nc1ccccc1NC(=O)c1ccc(C(=O)NCCN2CCC3(CCCCC3)CC2)cn1] | 1013 |
| 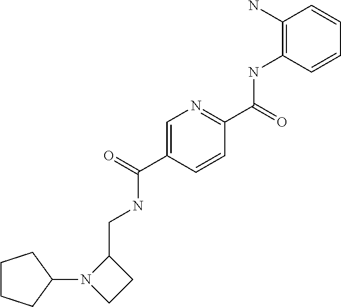 [SMILES: Nc1ccccc1NC(=O)c1ccc(C(=O)NCC2CCN2C2CCCC2)cn1] | 1015 |
| 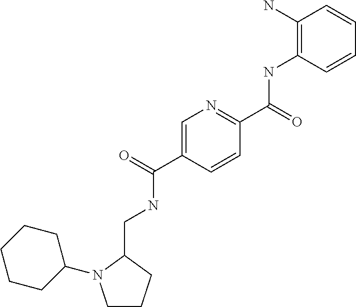 [SMILES: Nc1ccccc1NC(=O)c1ccc(C(=O)NCC2CCCN2C2CCCCC2)cn1] | 1046 |
| 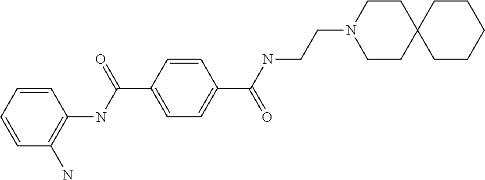 [SMILES: Nc1ccccc1NC(=O)c1ccc(C(=O)NCCN2CCC3(CCCCC3)CC2)cc1] | 1020 |
| 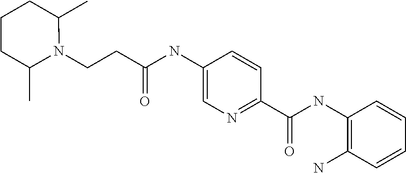 [SMILES: CC1CCCC(C)N1CCC(=O)Nc1ccc(C(=O)Nc2ccccc2N)nc1] | 1023 |
| 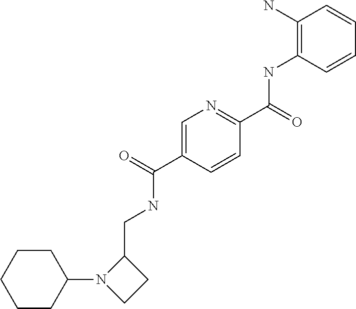 [SMILES: Nc1ccccc1NC(=O)c1ccc(C(=O)NCC2CCN2C2CCCCC2)cn1] | 1045 |
| 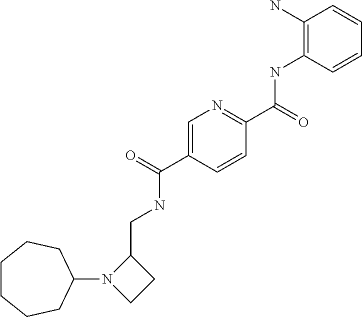 [SMILES: Nc1ccccc1NC(=O)c1ccc(C(=O)NCC2CCN2C2CCCCCC2)cn1] | 1050 |

In some cases, the compound as disclosed herein is one of Compounds 1009, 1011, 1013, 1015, 1020, 1045, 1046, 1050, 1023, 1038, 1039, or 1041, or a pharmaceutically acceptable salt thereof.

In some cases, the compound as disclosed herein is one of Compounds 1054, 1055, 1056, or 1057, or a pharmaceutically acceptable salt thereof.

The salts, e.g., pharmaceutically acceptable salts, of compounds disclosed herein can be prepared by reacting the appropriate base or acid with an appropriate amount of compound.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include anions, for example sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, and mandelate.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N [symbol] dibenzylethylenediamine, chloroprocaine, choline, diethanolamine, dicyclohexylamine, ethylenediamine, N-methylglucamine, and procaine.

### Synthesis of Compounds

For the synthesis of compounds several building blocks can be obtained following described in literature procedures. 6-[(2-{[(Tertbutoxy) carbonyl]amino}phenyl)carbamoyl]pyridine-3-carboxylic acid can be synthesized as reported in, e.g., Mahboobi et al, J. Med. Chem., 52:2265-2279, 2009, by basic hydrolysis of the corresponding ester obtained via coupling of 5-(methoxycarbonyl)pyridine-2-carboxylic acid with mono boc-protected 1-2-phenylenediamine. 4-[(2-{[(Tertbutoxy)carbonyl]amino}phenyl) carbamoyl]benzoic acid can be synthesized by the basic hydrolysis of ester obtained via reaction of 5-(methoxycarbonyl)benzene-2-carboxylic acid with mono Boc-protected 1-2-phenylenediamine. Target compounds can be synthesized by reaction of these acids with various amines in the presence of a coupling reagent (e.g., HATU/EDC) and a base (e.g., DIPEA). Further guidance for reaction conditions and sequence can be found in the Examples section below.

### Methods of Use

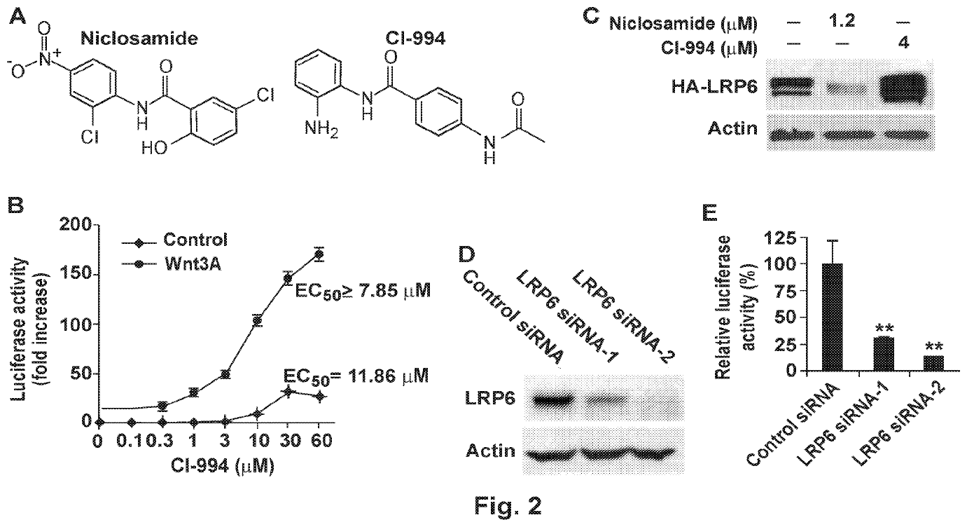

Niclosamide (5-chloro-N-[2-chloro-4-nitrophenyl]-2-hydroxy benzamide), an FDA approved antihelminthic drug, suppresses Wnt/β-catenin signaling by inducing Wnt co-receptor LRP6 degradation in cancer cells [35]. Interestingly, a very recent study reported that CI-994, an analog of niclosamide, is able to synergize with Wnt3A to activate the activity of the TCF/LEF reporter [36]. CI-994 is an orally active class I histone deacetylase (HDAC) inhibitor [37]. CI-994 increased Wnt reporter activity (FIG. 2B) and cytosolic free β-catenin level in HEK293 cells. However, other HDAC inhibitors including JNJ-26481585, PCI-34041, Droxinostat, Scriptaid and Panobinostat were unable to activate Wnt/β-catenin signaling in HEK293 cells, indicating that the stimulatory effect of CI-994 on Wnt/β-catenin signaling is unrelated to its inhibitory effect on HDACs. Mechanistically, CI-994 significantly enhanced Wnt co-receptor LRP6 expression in HT1080 fibrosarcoma cells stably transduced with HA-LRP6 (FIG. 2C), which is opposite to the effect of niclosamide on LRP6 expression (FIG. 2C) [35]. Moreover, knockdown of LRP6 expression significantly attenuated the effects of CI-994 on Wnt/β-catenin signaling in HEK293 cells (FIGS. 2D & 2E), indicating that CI-994 stimulates Wnt/β-catenin signaling by enhancing LRP6 expression.

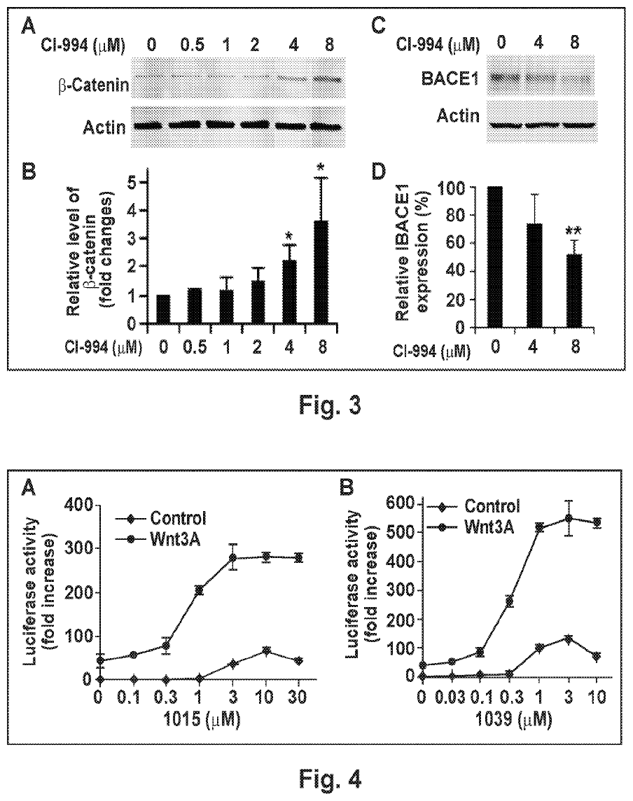

BACE1 is the enzyme that initiates the generation of Aβ, and is an attractive drug target for lowering cerebral Aβ levels for the treatment of AD [38, 39]. A recent study demonstrated that Wnt/β-catenin signaling plays an important role in BACE1 expression and Aβ production in neuronal cells [10]. It was found that β-catenin binds to TCF/LEF motifs located in the BACE1 promoter, and that activation of Wnt/β-catenin signaling represses the transcriptional activity of BACE1 and consequently inhibits Aβ production [10]. Consistently, we found that CI-994 significantly increased A-catenin levels and deceased BACE1 expression in neuronal N2a cells (FIG. 3).

The potency and selectivity of Wnt modulators was optimized, as well as drug-like properties, and a series of novel small molecule activators of Wnt/β-catenin signaling are disclosed herein. FIG. 4 shows activation of Wnt/β-catenin signaling by Wnt activator 1015 in HEK293 cells. Various compounds which display more potent activity than CI-994 in enhancing Wnt reporter Super8XTOPFlash activity in HEK293 cells are shown in Table A, and activity of some compounds in activating Wnt/β-catenin signaling in Wnt3A-expressing HEK293 cells are shown in the Examples.

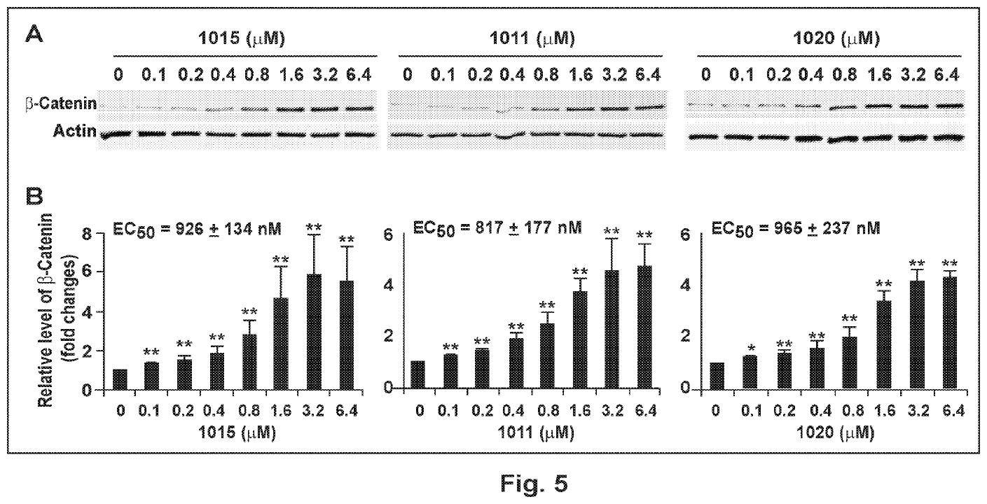

FIG. 5 shows that the lead compounds 1015, 1011 and 1020 significantly enhanced β-catenin level at the concentration as low as 0.1 μM in neuronal N2a cells with the EC₅₀ values (X±SD) of 0.96±0.14, 0.93±0.01 and 1.02±0.24 μM, respectively (FIG. 5), indicating that 1015, 1011 and 1020 are potent Wnt activators in activation of Wnt/β-catenin signaling in neuronal cells.

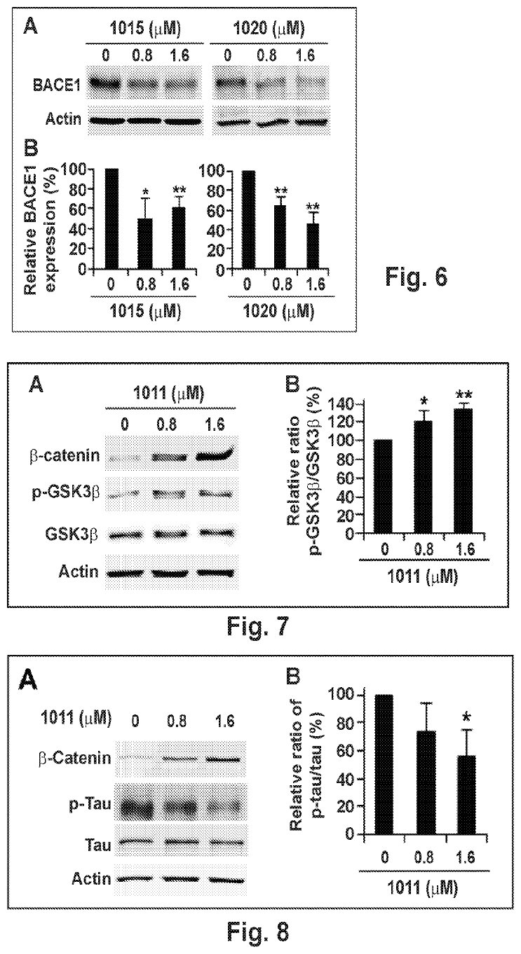

A recent study demonstrated that activation of Wnt/β-catenin signaling suppresses BACE1 expression in neuronal cells [10]. As shown in FIG. 6, 1015 and 1020 at 0.8 μM and 1.6 μM significantly inhibited BACE1 expression in N2a cells.

It has been established that Wnt proteins activate Wnt/β-catenin signaling by a mechanism that involves the inhibition of GSK3β, and that phosphorylation at Ser9 inhibits GSK3β activity [19, 40]. Indeed, we found that 1011 at 0.8 μM and 1.6 μM significantly enhanced GSK3β phosphorylation at Ser9 in N2a cells (FIG. 7), indicating that 1011 is able to suppress GSK3β activity in neuronal cells.

One of the two major hallmarks of AD is the presence of neurofibrillary tangles (NFTs), which are composed of hyperphosphorylated forms of the microtubule-associated protein tau in neurons [41, 42]. Tau phosphorylation by GSK3β potentially contributes to the pathology of neurodegeneration [43], and GSK3β has been targeted in clinical trials [44]. FIG. 8 shows that 1011 at 0.8 and 1.6 μM suppressed tau phosphorylation in N2a cells, further suggesting that the Wnt activators have great therapeutic potential for the treatment of AD.

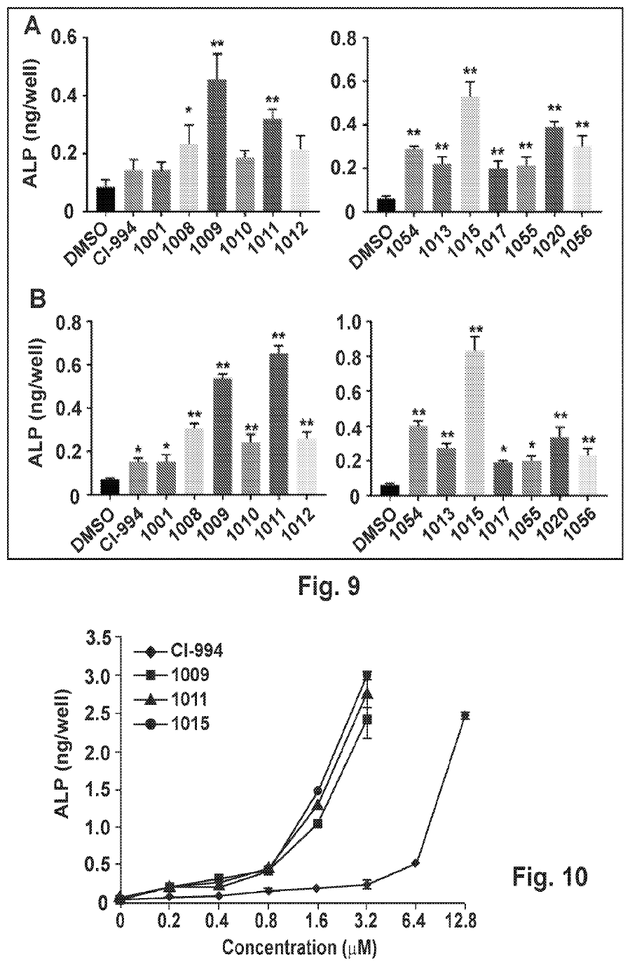

C2C12 cells are uncommitted mesenchymal progenitor cells that can be differentiated into osteoblasts upon activation of Wnt/β-catenin signaling [45, 46]. To determine whether the Wnt activators are able to induce osteoblastic differentiation, we examined the activity of alkaline phosphatase (ALP), a specific marker of osteoblast differentiation, in C2C12 cells. It was found that CI-994 and 13 compounds as disclosed herein at 0.8 μM and 1.6 μM significantly enhanced ALP activity in C2C12 (FIG. 9). Moreover, dose-response studies confirm that 1015, 1011 and 1009 are more potent than CI-994 in inducing C2C12 osteoblastic differentiation (FIG. 10). All together, these results indicate that the Wnt activators have great therapeutic potential for the treatment of osteoporosis and other bone disorders.

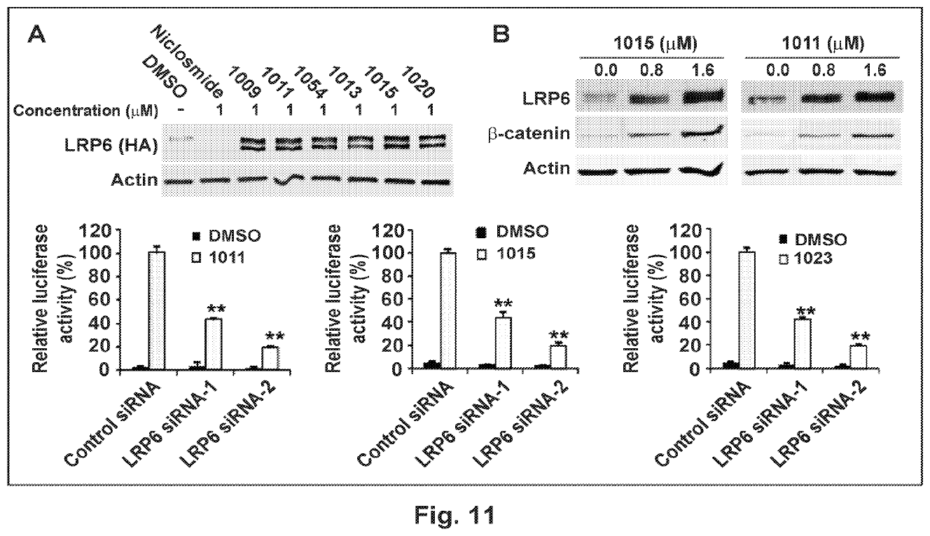

CI-994 and niclosamide display opposite effects on LRP6 expression, and CI-994 stimulates Wnt/β-catenin signaling by enhancing LRP6 expression (FIG. 2). As expected, the novel Wnt activators enhanced exogenous HA-LRP6 expression driven by CMV promoter in human fibrosarcoma cancer HT1080 cells (FIG. 11A). Moreover, 1015 and 1011 at 0.8 and 1.6 μM significantly enhanced endogenous LRP6 expression in N2a cells (FIG. 11B). In addition, knockdown of LRP6 expression significantly attenuated the effects of 1011, 1015 and 1023 on Wnt/β-catenin signaling in HEK293 cells (FIG. 11C). All together, these results indicate that the Wnt activators stimulate Wnt/β-catenin signaling via Wnt co-receptor LRP6.

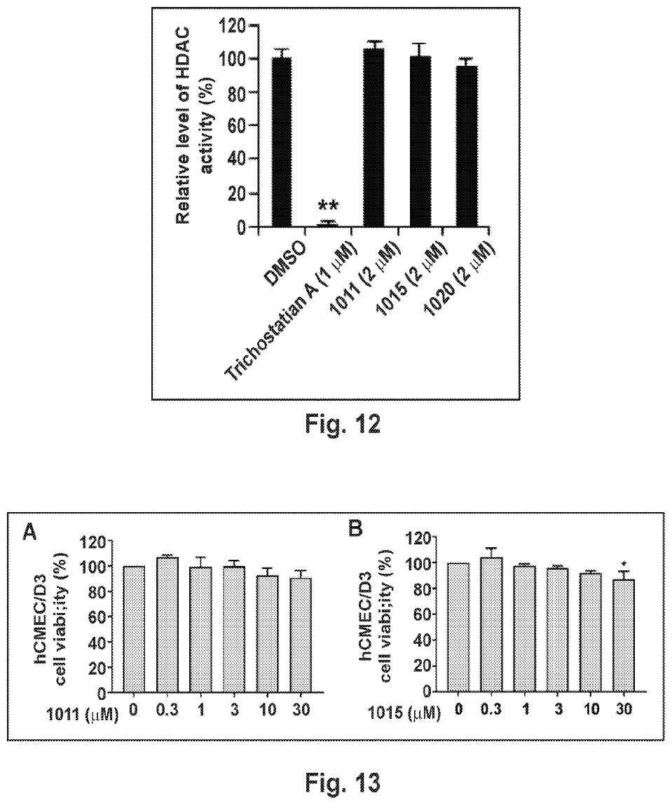

CI-994 is an orally active class I histone deacetylase (HDAC) inhibitor [37]. As such, the Wnt activators disclosed herein may retain inhibitory activity against HDACs. Interestingly, HDAC is also an attractive therapeutic target of AD, although the molecular mechanisms are unclear [47, 48]. 1015, 1011 and 1020 do not display any inhibitory effects on HDAC activity at 2 μM (FIG. 12), suggesting that the stimulating effects of Wnt activators on Wnt/β-catenin signaling is not associated with HDAC activity.

To determine potential cytotoxicity of Wnt activators, cell viability assay was performed at increasing dosages of compounds (0.3, 1, 3, 10 and 30 μM). 1011 is not toxic at all the tested concentrations, and 1015 is not toxic at or below the 10 μM concentration to human endothelial capillary hCMEC/D3 cells (FIG. 13). These results indicate that both 1011 and 1015 have very low cytotoxicity for brain endothelial cells.

The bacterial reverse mutation test uses amino acid requiring strains of Salmonella typhimuium and Escherichia coli (E. coli) to detect chemical substances that can produce genetic damage and lead to gene mutations. The mutagenic potential of 1011 and 1015 and their metabolites was assessed by exposing bacterial strains to the compounds and evaluating at 400 μM, and evaluating the numbers of revertant colonies grown in the absence (trace quantities) of the amino acid. 1011 and 1015 show no cytotoxicity for bacterial cells. Importantly, the test compounds and their metabolites have no mutagenic effects on all tested strains of S. typhimuium and E. coli (see Tables in Examples).

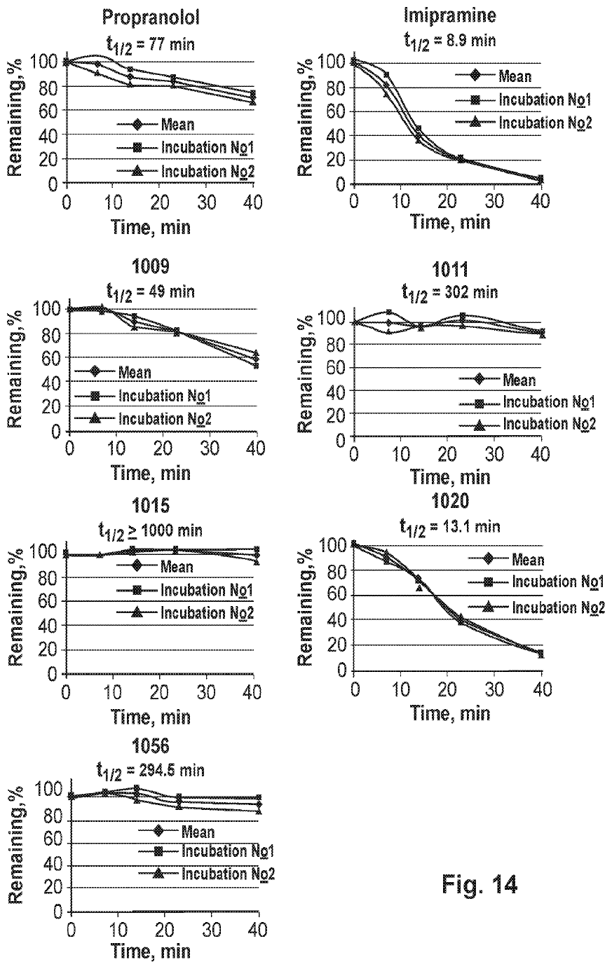

By performing kinetic solubility assays, all tested compounds displayed high aqueous solubility (≥194 μM at pH 7.4) (See Table in Examples). Moreover, the microsomal metabolic stability assays revealed that 1011 and 1015 and 1056 exhibited good stability in mouse hepatic microsomal system (FIG. 14). 1009 showed moderate stability, and 1020 was unstable in mouse hepatic microsomal test system. “No cofactor” control data indicated that the observed instability is primarily determined by CYP450 activity. The high aqueous solubility and microsomal metabolic stability of 1011 and 1015 indicate these compounds would be orally bioavailable and efficacious in vivo.

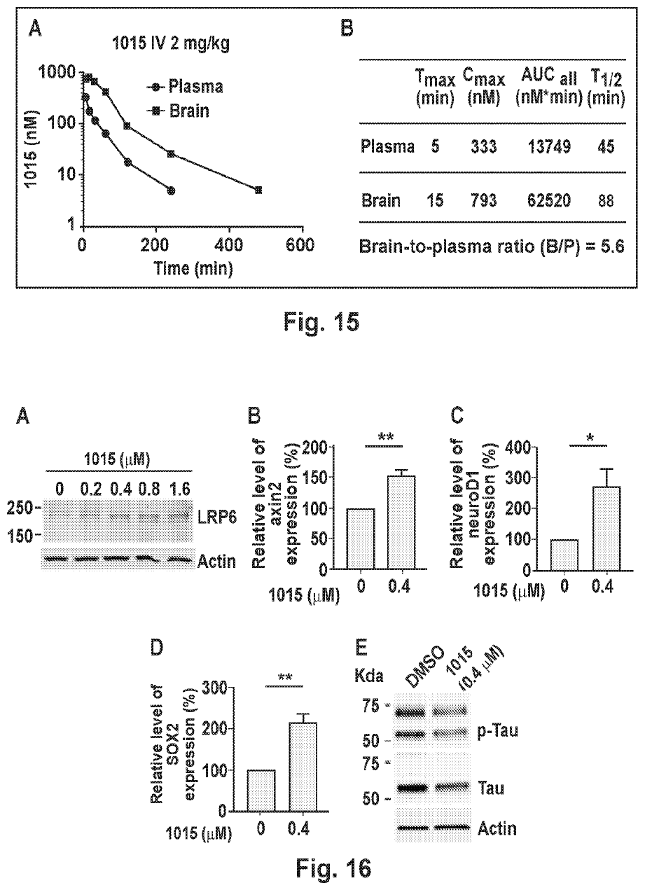

Due to minimal blood brain barrier (BBB) transport of many potential CNS drugs, there are few effective treatments for the majority of CNS disorders [49]. 1015 displays a great brain penetration. After intravenous (IV) administration (2 mg/kg) of 1015 in male Balb/c mice, the compound rapidly entered into the brain, and displayed a much higher concentration in brain than in plasma. The brain concentration/plasma concentration ratio (B/P) was 5.6 after 30 min IV injection of 1015 (FIG. 15). Moreover, the half-life (t_{1/2}) of 1015 is much longer in brain than in plasma (FIG. 15). Altogether, these findings indicate that 1015 has a great potential to be developed as a CNS drug.

CI-994 [4-(acetylamino)-N-(2-aminophenyl)benzamide] was found to be a Wnt activator by enhancing Wnt co-receptor LRP6 expression. With the optimization of CI-994, a series of novel potent Wnt activators were developed, with at least nine compounds exhibiting EC₅₀ values less than 1 μM in activation of Wnt/β-catenin signaling in the cell-based assay. Moreover, 1015 displays a great brain penetration with very low cytotoxicity and no genetic toxicity. Altogether, these findings indicate that compounds disclosed herein have a great potential as a first-in-class drug for AD, osteoporosis and other diseases associated with the dysregulation of Wnt signaling.

Provided herein, therefore, are methods of modulating Wnt/β-catenin signaling pathway using a compound or salt thereof as disclosed herein. In various cases, the compounds disclosed herein can be used to treat a neurological disorder in a subject. In some cases, the neurological disorder is selected from the group consisting of Alzheimers disease, frontotemporal dementias, dementia with lewy bodies, a prion disease, Parkinsons disease, Huntingtons disease, progressive supranuclear palsy, corticobasal degeneration, multiple system atrophy, amyotrophic lateral sclerosis (ALS), inclusion body myositis, autism, degenerative myopathy, diabetic neuropathy, endocrine neuropathy, orthostatic hypotension, multiple sclerosis and Charcot-Marie-Tooth disease. In various cases, the compounds disclosed herein can be used to treat bone degeneration in a subject.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound as disclosed herein to an individual in need of such treatment.

The term “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein or known in the art. The precise effective amount for a subject will depend upon the subjects body weight, size, and health; the nature and extent of the condition; and the therapeutic or combination of therapeutics selected for administration. Therapeutically effective amounts for a given situation can be determined by routine experimentation that is within the skill and judgment of the clinician.

Presented herein is a method of administering a compound as disclosed herein or salt thereof as the neat compound or as a pharmaceutical composition orally, intravenously, or parenterally. In some cases, the compound or salt thereof is administered orally. Administration of a pharmaceutical composition, or neat compound, can be performed during or after the onset of the disease or condition of interest. Typically, the pharmaceutical compositions are sterile, and contain no toxic, carcinogenic, or mutagenic compounds that would cause an adverse reaction when administered.

### Dosing and Pharmaceutical Formulations

Dosages of the compounds disclosed herein can be administered as a dose measured in mg/kg. Contemplated mg/kg doses of the disclosed compounds include about 0.001 mg/kg to about 1000 mg/kg. Specific ranges of doses in mg/kg include about 0.1 mg/kg to about 500 mg/kg, about 0.5 mg/kg to about 200 mg/kg, about 1 mg/kg to about 100 mg/kg, about 1 mg/kg to about 50 mg/kg, about 1 mg/kg to about 40 mg/kg, and about 5 mg/kg to about 30 mg/kg.

A compound used in a method described herein can be administered in an amount of about 0.005 to about 750 milligrams per dose, about 0.05 to about 500 milligrams per dose, or about 0.5 to about 250 milligrams per dose. For example, a compound can be administered, per dose, in an amount of about 0.005, 0.05, 0.5, 1, 2, 3, 4, 5, 10, 15, 20, 30, 40, 50, 100, 150, 200, 250, 300, 350, 400, 450, 500, 550, 600, 650, 700, or 750 milligrams, including all doses between 0.005 and 750 milligrams.

As herein, the compounds described herein may be formulated in pharmaceutical compositions with a pharmaceutically acceptable excipient, carrier, or diluent. The compound or composition comprising the compound is administered by any route that permits treatment of the disease or condition. One route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, topically, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or a compound as disclosed herein can be administered over a period of time, either in divided doses or in a continuous-release formulation or administration method (e.g., a pump). However the compounds disclosed herein are administered to the subject, the amounts of compound administered and the route of administration chosen should be selected to permit efficacious treatment of the disease or condition.

In an embodiment, the pharmaceutical compositions are formulated with one or more pharmaceutically acceptable excipient, such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. More particularly, the pharmaceutical compositions may comprise a therapeutically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of a disorder as disclosed herein.

Formulations, e.g., for parenteral or oral administration, are most typically solids, liquid solutions, emulsions or suspensions, while inhalable formulations for pulmonary administration are generally liquids or powders. A pharmaceutical composition can also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington [symbol] Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringers solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In some embodiments, the compounds described herein may be formulated for oral administration in a lipid-based formulation suitable for low solubility compounds. Lipid-based formulations can generally enhance the oral bioavailability of such compounds.

As such, pharmaceutical compositions comprise a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

### Examples

Compounds were generally made via the following synthetic protocols.

Acylation with EDC: The vial was charged with DMF solutions of acid (typically 0.1 mmol, 1 eq.), amine (1.1 eq.), EDC base (1.05 eq.), HOAt (1.05 eq.) and DIPEA* (2 eq.). The reaction mixture was left to stand overnight at ambient temperature. The solvent was evaporated and the residue was dissolved in DMSO and subjected to HPLC purification. The purification was performed using Agilent 1260 Infinity systems equipped with DAD and mass-detector. Waters Sunfire C18 OBD Prep Column, 100 Å, 5 μm, 19 mm×100 mm with SunFire C18 Prep Guard Cartridge, 100 Å, 10 μm, 19 mm×10 mm was used. Deionized Water (phase A) and HPLC-grade Methanol (phase B) were used as an eluent. In some cases, ammonia or TFA was used as an additive to improve the separation of the products. In these cases, free bases and TFA salts of the products were formed respectively. In case of using a salt of the reagent, an additional amount of DIPEA was added to the reaction mixture to transfer the amine to the base form.

Acylation and Boc-deprotection with HATU: The vial was charged with DMF solutions of acid (typically 0.1 mmol, 1.1 eq.), amine (1 eq.), HATU (1.05 eq.), and DIPEA* (2.5 eq.). The reaction mixture was left to stand overnight. Then the solvent was evaporated and 1 ml of cleavage cocktail [TFA (92.5% v/v), water (5% v/v) and TIPS (2.5% v/v)] was added. The mixture was left to react for 4h and evaporated to dryness. The residue was dissolved in DMSO and subjected to HPLC purification. The purification was performed using Agilent 1260 Infinity systems equipped with DAD and mass-detector. Waters Sunfire C18 OBD Prep Column, 100 Å, 5 μm, 19 mm×100 mm with SunFire C18 Prep Guard Cartridge, 100 Å, 10 μm, 19 mm×10 mm was used. Deionized Water (phase A) and HPLC grade Methanol (phase B) were used as an eluent. In some cases, ammonia or TFA was used as an additive to improve the separation of the products. In these cases, free bases and TFA salts of the products were formed respectively. In case of using a salt of the reagent, an additional amount of DIPEA was added to the reaction mixture to transfer the amine to the base form.

Synthesis of 1001, 1002, 1003, 1004, 1005, 1006, 1007, 1008, 1009, 1010, 1011, 1015, 1012, 1013, 1014, 1016, 1017, 1018, 1028, 1029, 1030, 1031, 1032, 1033, 1034, 1035, 1036, 1037, 1038, 1039, 1040, 1041, 1042, 1045, 1046, 1047, 1048, 1049, 1050, and 1051 was carried as shown in Scheme 1. The final compounds were obtained by parallel chemistry exploiting the general procedure Acylation-boc-deprotection: HATU described above. Chiral separation was performed by HPLC with the use of CHIRALPAK chiral columns using a water/methanol mobile phase.

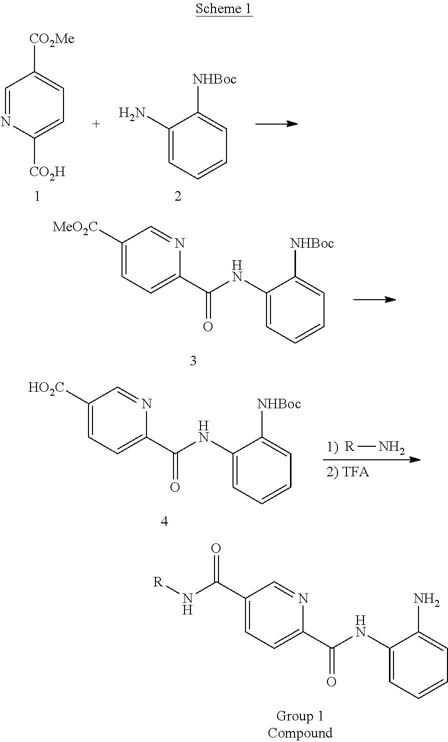

1001: ¹H NMR (DMSOd6): δ=10.12 (s, 1H, NH), 9.09 (s, 1H, C_{6pyr}H), 8.85 (m, 1H, NH), 8.40 (m, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 2.54 (m, 8H, 4NCH₂), 1.69 (m, 2H, CH₂), 1.55 (m, 8H, CH_{2azepane}) ppm. LS-MS (m/z): calcd./found for [M+H]⁺ 396.24/396.2.

1002: ¹H NMR (DMSOd6): δ=10.13 (s, 1H, NH), 9.08 (s, 1H, C_{6pyr}H), 8.63 (d, 1H, NH), 8.42 (d, 1H, C H), 8.21 (d, 1H, C_{3pyr}H), 7.48 (d, 1H, C_{Ar}H), 6.97 (t, 1H, C_{Ar}H), 6.83 (d, 1H, C_{Ar}H), 6.65 (t, 1H, C_{Ar}H), 4.92 (s, 2H, NH₂), 3.79 (m, 1H, CH), 2.89 (d, 2H, NCH₂), 2.05 (m, 7H, CH, 2CH₂), 1.49 (m, 12H, 6CH₂) ppm. LS-MS (m/z): calcd./found for [M+H]⁺ 422.25/422.2.

1003: ¹H NMR (DMSOd6): δ=10.13 (s, 1H, NH), 9.08 (s, 1H, C_{6pyr}H), 8.65 (d, 1H, NH), 8.42 (d, 1H, C H), 8.21 (d, 1H, C_{3pyr}H), 7.48 (d, 1H, C_{Ar}H), 6.97 (t, 1H, C_{Ar}H), 6.83 (d, 1H, C_{Ar}H), 6.66 (t, 1H, C_{Ar}H), 6.16 (t, 1H, CHF₂), 4.92 (s, 2H, NH₂), 3.80 (d, 2H, CH_{2acyclic}), 2.93 (d, 2H, CH₂), 2.50 (m, 4H, 2NCH_{2 cyclic}), 1.71 (m, 4H, 2CH₂) ppm. LS-MS (m/z): calcd./found for [M+H]⁺ 404.19/404.1.

1004: ¹H NMR (DMSOd6): δ=10.12 (s, 1H, NH), 9.09 (s, 1H, C_{6pyr}H), 8.83 (t, 1H, NH), 8.41 (d, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.92 (s, 2H, NH₂), 3.21 (m, 2H, CH_{2acyclic}), 2.23 (m, 4H, 2CH₂), 1.99 (m, 2H, CH₂), 1.44 (m, 1H, CH_{acyclic}), 1.43 (m, 5H, CH, 2CH_{2cyclic}), 0.83 (m, 6H, 2CH₃) ppm. LS-MS (m/z): calcd./found for [M+H]⁺ 410.25/410.2.

1005: ¹H NMR (DMSOd6): δ=10.08 (s, 1H, NH), 8.70 (d, 1H, C_{4pyr}H), 8.18 (d, 1H, C_{3pyr}H), 8.04 (dd, 1H, NH), 7.48 (d, 1H, C_{Ar}H), 6.97 (t, 1H, C_{Ar}H), 6.82 (d, 1H, C_{Ar}H), 6.65 (t, 1H, C_{Ar}H), 4.93 (s, 2H, NH₂), 3.37 (m, 4H, CH_{2cyclic}, CH_{2acyclic}), 2.29 (m, 5H, NCH₃, CH₂), 1.48 (m, 4H, 2CH₂), 0.85 (d, 3H, 2CH₃) ppm. LS-MS (m/z): calcd./found for [M+H]⁺ 382.22/382.2.

1006: ¹H NMR (DMSOd6): δ=10.12 (s, 1H, NH), 9.10 (s, 1H, C_{6pyr}H), 8.71 (m, 1H, NH), 8.42 (d, 1H, C_{4pyr}H), 8.21 (d, 1H, C_{3pyr}H), 7.48 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 3.24 (d, 2H, CH_{2acyclic}), 2.74 (m, 4H, 2NCH_{2cyclic}), 1.69 (m, 8H, 4CH_{2cyclic}) ppm. LS-MS (m/z): calcd./found for [M+H]⁺ 380.2/380.2.

1007: ¹H NMR (DMSOd6): δ=10.13 (s, 1H, NH), 9.08 (s, 1H, C_{6pyr}H), 8.55 (d, 1H, NH), 8.42 (d, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 3.91 (m, 1H, NCH), 2.51 (m, 5H, CH, 2CH₂), 1.64 (m, 14H, 7CH₂) ppm. LS-MS (m/z): calcd./found for [M+H]⁺ 422.25/422.2.

1008: ¹H NMR (DMSOd6): δ=10.13 (s, 1H, NH), 9.10 (m, 1H, C_{6pyr}H), 8.90 (dd, 1H, NH), 8.45 (m, 1H, C_{4pyr}H), 8.20 (t, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 4.35 (m, 1H, NCH), 2.95 (m, 1H, NCH), 1.73 (m, 12H, 6CH₂) ppm. LS-MS (m/z): calcd./found for [M+H]⁺ 380.2/380.2.

1009: ¹H NMR (DMSOd6): δ=10.13 (s, 1H, NH), 9.08 (s, 1H, C_{6pyr}H), 8.80 (t, 1H, NH), 8.40 (d, 1H, C_{4pyr}H), 8.21 (d, 1H, C_{3pyr}H), 7.46 (d, 1H, C_{Ar}H), 6.97 (t, 1H, C_{Ar}H), 6.82 (d, 1H, C_{Ar}H), 6.65 (t, 1H, C_{Ar}H), 4.93 (s, 2H, NH₂), 3.36 (m, 2H, NCH_{2acyclic}), 2.85 (m, 2H, CH_{2acyclic}), 2.32 (m, 3H, CH, CH₂), 1.47 (m, 13H, CH, 6CH₂) ppm. LS-MS (m/z): calcd./found for [M+H]⁺ 422.25/422.0.

1010: ¹H NMR (DMSOd6): δ=10.13 (s, 1H, NH), 9.10 (s, 1H, C_{6pyr}H), 8.87 (d, 1H, NH), 8.44 (d, 1H, C_{4pyr}H), 8.20 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.92 (s, 2H, NH₂), 4.40 (m, 1H, NCH_{cyclic}), 2.65 (m, 1H, CH_{cyclic}), 2.48 (m, 6H, 3CH₂), 1.59 (m, 8H, 4CH₂) ppm. LS-MS (m/z): calcd./found for [M+H]⁺ 394.2/394.2.

1012: ¹H NMR (DMSOd6): δ=10.13 (s, 1H, NH), 9.10 (s, 1H, C_{6pyr}H), 8.86 (d, 1H, NH), 8.44 (m, 1H, C_{4pyr}H), 8.20 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.92 (s, 2H, NH₂), 4.40 (m, 1H, NCH_{cyclic}), 2.54 (m, 1H, CH_{cyclic}), 2.44 (m, 4H, 2NCH₂), 1.47 (m, 12H, 6CH₂) ppm. LS-MS (m/z): calcd./found for [M+H]⁺ 408.24/408.5.

1013: ¹H NMR (DMSOd6): δ=10.12 (s, 1H, NH), 9.08 (s, 1H, C_{6pyr}H), 8.79 (d, 1H, NH), 8.40 (m, 1H, C_{4pyr}H), 8.21 (d, 1H, C_{3pyr}H), 7.47 (d, 1H, C_{Ar}H), 6.97 (t, 1H, C_{Ar}H), 6.82 (d, 1H, C_{Ar}H), 6.65 (t, 1H, C_{Ar}H), 4.93 (s, 2H, NH₂), 3.41 (m, 2H, N_{acyl} CH_{2acyclic}), 2.38 (m, 6H, 3NCH₂), 1.35 (m, 14H, 7CH₂) ppm. LS-MS (m/z): calcd./found for [M+H]⁺ 436.27/436.0.

1014: ¹H NMR (DMSOd6): δ=10.13 (s, 1H, NH), 9.10 (s, 1H, C_{6pyr}H), 8.90 (d, 1H, NH), 8.44 (m, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 6.12 (t, 1H, CHF₂), 4.93 (s, 2H, NH₂), 4.39 (m, 1H, NCH_{cyclic}), 2.85 (m, 4H, 2CH₂), 2.62 (m, 2H, NCH_{2cyclic}), 1.98 (m, 2H, NCH_{2acyclic}) ppm. LS-MS (m/z): calcd./found for [M+H]⁺ 390.2/390.0.

1016: ¹H NMR (DMSOd6): δ=10.01 (s, 1H, NH), 9.08 (s, 1H, C_{6pyr}H), 8.40 (m, 2H, C_{4pyr}H, NH), 8.22 (d, 1H, C_{3pyr}H), 7.52 (d, 1H, C_{Ar}H), 6.94 (t, 1H, C_{Ar}H), 6.83 (d, 1H, C_{Ar}H), 6.66 (t, 1H, C_{Ar}H), 4.69 (s, 2H, NH₂), 3.99 (m, 1H, N_{acyl}CH_{cyclic}), 3.13 (q, 2H, CH₂CF₃), 3.07 (m, 1H, NCH_{2cyclic}), 2.88 (m, 1H, NCH_{2cyclic}), 2.39 (m, 2H, NCH_{2cyclic}), 1.82 (m, 2H, CH_{2acyclic}), 1.49 (m, 2H, CH_{2acyclic}) ppm. LS-MS (m/z): calcd./found for [M+H]⁺ 422.18/422.2.

1017: ¹H NMR (DMSOd6): δ=10.13 (s, 1H, NH), 9.10 (s, 1H, C_{6pyr}H), 8.90 (d, 1H, NH), 8.44 (d, 1H, C_{4pyr}H), 8.20 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 4.42 (m, 1H, N_{acyl}CH_{cyclic}), 2.71 (t, 1H, NCH_{2acyclic}), 2.62 (t, 1H, NCH_{2cyclic}), 2.49 (m, 1H, NCH_{2cyclic}), 2.42 (m, 1H, NCH_{2cyclic}), 2.27 (s, 3H, NCH₃), 2.20 (m, 1H, CH_{2cyclic}), 1.80 (m, 1H, CH_{2cyclic}) ppm. LS-MS (m/z): calcd./found for [M+H]⁺ 340.17/340.2.

1018: ¹H NMR (DMSOd6): δ=10.13 (s, 1H, NH), 9.10 (s, 1H, C_{6pyr}H), 8.68 (d, 1H, NH), 8.45 (d, 1H, C_{4pyr}H), 8.22 (d, 1H, C_{3pyr}H), 7.49 (d, 1H, C_{Ar}H), 6.97 (t, 1H, C_{A}—H), 6.83 (d, 1H, C_{Ar}H), 6.66 (t, 1H, C_{Ar}H), 4.93 (s, 2H, NH₂), 3.99 (d, 1H, N_{acyl}CH_{cyclic}), 3.15 (m, 1H, NCH_{2cyclic}), 2.87 (m, 1H, NCH_{2cyclic}), 2.71 (m, 4H, 2NCH_{2cyclic}), 1.90 (m, 1H, CH_{cyclic}), 1.83 (m, 1H, CH_{cyclic}), 1.59 (m, 2H, CH_{2cyclic}), 1.35 (m, 1H, CH_{2cyclic}) ppm. LS-MS (m/z): calcd./found for [M+H]⁺ 366.19/366.1.

1028: LS-MS (m/z): calcd./found for [M+H]⁺ 368.2/368.2.

1029: LS-MS (m/z): calcd./found for [M+H]⁺ 392.2/392.2.

1030: LS-MS (m/z): calcd./found for [M+H]⁺ 398.21/398.2.

1031: LS-MS (m/z): calcd./found for [M+H]⁺ 404.19/404.2.

1032: LS-MS (m/z): calcd./found for [M+H]⁺ 410.21/410.2.

1033: LS-MS (m/z): calcd./found for [M+H]⁺ 416.2/416.2.

1034: LS-MS (m/z): calcd./found for [M+H]⁺ 416.2/416.2.

1035: LS-MS (m/z): calcd./found for [M+H]⁺ 420.21/420.2.

1036: LS-MS (m/z): calcd./found for [M+H]⁺ 422.18/422.2.

1037: LS-MS (m/z): calcd./found for [M+H]⁺ 430.22/430.2.

1038: LS-MS (m/z): calcd./found for [M+H]⁺ 436.27/436.2

1039: LS-MS (m/z): calcd./found for [M+H]⁺ 444.24/444.2.

1040: LS-MS (m/z): calcd./found for [M+H]⁺ 444.24/444.2.

1041: LS-MS (m/z): calcd./found for [M+H]⁺ 446.21/446.2.

1042: LS-MS (m/z): calcd./found for [M+H]⁺ 453.2/453.2.

1045: ¹H NMR (DMSOd6): δ=10.12 (s, 1H, NH), 9.10 (s, 1H, C_{6pyr}H), 8.88 (s, 1H, NH), 8.41 (m, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.92 (s, 2H, NH₂), 3.54 (m, 1H, NCH_{cyclic}), 3.25 (m, 4H, N_{acyl}CH_{2acyclic}, NCH_{2cyclic}) 2.70 (m, 1H, NCH_{cyclic}), 2.04 (m, 2H, CH_{2cyclic}), 1.81 (m, 2H, CH_{2cyclic}), 1.59 (s, 4H, 2CH_{2cyclic}), 1.17 (m, 2H, CH_{2cyclic}), 1.01 (m, 2H, CH_{2cyclic}) ppm. LS-MS (m/z): calcd./found for [M+H]⁺ 408.22/408.2.

1046: LS-MS (m/z): calcd./found for [M+H]⁺ 422.25/422.2.

1047: LS-MS (m/z): calcd./found for [M+H]⁺ 396.24/396.2.

1048: LS-MS (m/z): calcd./found for [M+H]⁺ 404.19./404.2.

1049: LS-MS (m/z): calcd./found for [M+H]⁺ 450.28/450.2.

1050: LS-MS (m/z): calcd./found for [M+H]⁺ 422.25/422.2.

1051: LS-MS (m/z): calcd./found for [M+H]⁺ 396.24/396.2.

1011: ¹H NMR (DMSOd6): δ=10.13 (s, 1H, NH), 9.09 (s, 1H, C_{6pyr}H), 8.86 (m, 1H, NH), 8.40 (m, 1H, C_{4pyr}H), 8.22 (d, 1H, C_{3pyr}H), 7.46 (d, 1H, C_{Ar}H), 6.97 (t, 1H, C_{Ar}H), 6.82 (d, 1H, C_{Ar}H), 6.65 (t, 1H, C_{Ar}H), 4.93 (s, 2H, NH₂), 2.61 (m, 6H, 2CH, 2CH₂), 1.49 (m, 6H, 3CH₂), 1.12 (d, 6H, 2CH₃) ppm. LS-MS (m/z): calcd./found for [M+H]⁺ 396.23/396.0.

1015: ¹H NMR (DMSOd6): δ=10.13 (s, 1H, NH), 9.09 (s, 1H, C_{6pyr}H), 8.83 (m, 1H, NH), 8.41 (m, 1H, C_{4pyr}H), 8.22 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 3.52 (m, 1H, CH), 3.23 (m, 2H, CH₂), 2.98 (m, 2H, CH₂), 2.72 (m, 2H, CH₂), 1.53 (m, 9H, CH, 4CH₂) ppm. LS-MS (m/z): calcd./found for [M+H]⁺ 394.22/394.0.

1015-E1: ¹H NMR (DMSOd6): δ=10.32 (s, 1H, NH), 9.09 (s, 1H, C_{6pyr}H), 8.86 (m, 1H, NH), 8.42 (d, 1H, C_{4pyr}H), 8.24 (d, 1H, C_{3pyr}H), 7.78 (t, 1H, C_{Ar}H), 7.23 (d, 1H, C_{Ar}H), 6.97 (t, 1H, C_{Ar}H), 6.78 (d, 1H, C_{Ar}H), 6.58 (t, 1H, C_{Ar}H), 4.92 (s, 2H, NH₂), 3.56 (m, 5H, NCH_{2cyclic}, NCH_{2acyclic}, NCH_{cyclic}), 1.62 (m, 11H, CH, 5CH₂) ppm. LS-MS (m/z): calcd./found for [M+H]⁺ 394.21/394.2.

1015-E2: ¹H NMR (DMSOd6): δ=10.32 (s, 1H, NH), 9.09 (s, 1H, C_{6pyr}H), 8.86 (m, 1H, NH), 8.42 (d, 1H, C_{4pyr}H), 8.24 (d, 1H, C_{3pyr}H), 7.78 (t, 1H, C_{Ar}H), 7.23 (d, 1H, C_{Ar}H), 6.97 (t, 1H, C_{Ar}H), 6.78 (d, 1H, C_{Ar}H), 6.58 (t, 1H, C_{Ar}H), 4.92 (s, 2H, NH₂), 3.56 (m, 5H, NCH_{2cyclic}, NCH_{2acyclic}, NCH_{cyclic}), 1.62 (m, 11H, CH, 5CH₂) ppm. LS-MS (m/z): calcd./found for [M+H]⁺ 394.21/394.2.

To obtain 1043 and 1051, a synthesis was performed as outlined in Scheme 2. After with the use of general procedure Acylation: HATU, the final compounds were synthesized.

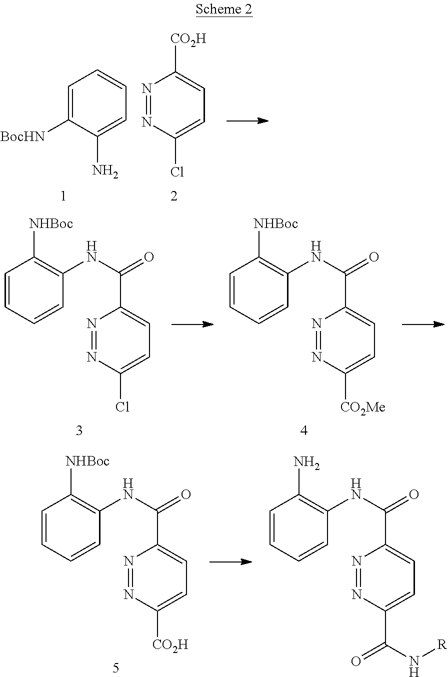

1043: ¹H NMR (DMSOd6): δ=10.18 (s, 1H, NH), 9.18 (m, 1H, NH), 8.44 (m, 2H, C_{pyridazine}H, 7.41 (d, 1H, C_{Ar}H), 7.00 (t, 1H, C_{Ar}H), 6.82 (d, 1H, C_{Ar}H), 6.65 (t, 1H, C_{Ar}H), 5.01 (s, 2H, NH₂), 3.59 (m, 1H, NCH_{cyclic}), 3.39 (m, 2H, N_{acyl}CH_{2acyclic}), 3.23 (m, 1H, NCH_{cyclic}), 2.75 (m, 2H, NCH_{2cyclic}), 1.89 (m, 2H, CH_{2cyclic}), 1.54 (d, 6H, 3CH_{2cyclic}), 1.32 (d, 2H, CH_{2cyclic}) ppm. LS-MS (m/z): calcd./found for [M+H]⁺ 395.22/395.2.

1051: ¹H NMR (CDCl₃): δ=9.92 (s, 1H, NH), 8.54 (m, 3H, 3CH_{Ar}), 7.54 (m, 1H, NH), 7.13 (d, 1H, CH_{Ar}), 6.89 (t, 2H, 2CH_{Ar}), 3.92 (s, 2H, NH₂), 3.65 (m, 2H, NCH_{2acyclic}), 2.63 (t, 2H, CH_{2acyclic}), 2.45 (m, 4H, 2NCH_{2cyclic}), 1.47 (m, 14H, 7CH_{2cyclic}) ppm. LS-MS (m/z): calcd./found for [M+H]⁺ 437.26/437.4.

A comparable synthesis as shown above in Scheme 2 with appropriate starting materials was used to prepare 1021, 1022, 1025, 1052, and 1053.

1021: ¹H NMR (CDCl3): δ=7.99 (d, 2H, C_{Ar}H), 7.94 (d, 3H, C_{Ar}H, NH), 7.38 (d, 1H, C_{Ar}H), 7.25 (s, 1H, NH), 7.11 (t, 1H, C_{Ar}H), 6.87 (m, 2H, C_{Ar}H), 3.86 (s, 2H, NH₂), 3.61 (m, 2H, 2NCH_{cyclic}), 3.30 (m, 2H, NCH_{2cyclic}), 2.84 (d, 2H, N_{acyl}CH_{2acyclic}), 1.97 (d, 2H, CH_{2cyclic}), 1.59 (d, 8H, 4CH_{2cyclic}) ppm. LS-MS (m/z): calcd./found for [M+H]⁺ 392.22/392.2.

1022: LS-MS (m/z): calcd./found for [M+H]⁺ 395.22/395.2.

1025: ¹H NMR (DMSOd6): δ=10.32 (s, 1H, NH), 8.80 (s, 1H, C_{6pyr}H), 8.52 (m, 1H, NH), 8.19 (m, 2H, C_{4pyr}H, C_{3pyr}H), 7.75 (d, 2H, C_{Ar}H), 7.21 (t, 2H, C_{Ar}H), 6.76 (d, 1H, C_{Ar}H), 6.55 (t, 1H, C_{Ar}H), 6.48 (s, 2H, NH2), 3.47 (m, 1H, NCH_{cyclic}), 3.22 (t, 3H, CH₂, CH), 2.70 (m, 2H, NCH_{2cyclic}), 1.61 (m, 10H, CH_{2cyclic}) ppm. LS-MS (m/z): calcd./found for [M+H]⁺ 394.21/394.2.

1052: ¹H NMR (DMSOd6): δ=10.19 (s, 1H, NH), 9.27 (m, 2H, C_{pyrazine}H), 8.94 (t, 1H, NH), 7.36 (d, 1H, C_{Ar}H), 6.99 (t, 1H, C_{Ar}H), 6.81 (d, 1H, C_{Ar}H), 6.63 (t, 1H, C_{Ar}H), 4.98 (s, 2H, NH₂), 3.44 (m, 2H, N_{acyl}CH_{2acyclic}), 2.50 (m, 2H, NCH_{2acyclic}), 2.37 (m, 4H, 2NCH_{2cyclic}), 1.37 (m, 10H, 5CH_{2cyclic}), 1.28 (m, 4H, 2CH_{2cyclic}) ppm. LS-MS (m/z): calcd./found for [M+H]⁺ 437.26/437.2.

1053: ¹H NMR (DMSOd6): δ=9.79 (s, 1H, NH), 8.31 (t, 1H, NH), 7.87 (m, 2H, 2C_{Ar}H), 7.73 (t, 1H, C_{Ar}H), 7.15 (d, 1H, C_{Ar}H), 6.99 (t, 1H, C_{Ar}H), 6.78 (d, 1H, C_{Ar}H), 6.59 (t, 1H, C_{Ar}H), 4.96 (s, 2H, NH₂), 3.36 (m, 2H, N_{acyl}CH_{2acyclic}), 2.46 (m, 2H, NCH_{2acyclic}), 2.38 (m, 4H, 2NCH_{2cyclic}), 1.38 (m, 10H, 5CH_{2cyclic}), 1.29 (t, 4H, 2CH_{2cyclic}) ppm. LS-MS (m/z): calcd./found for [M+H]⁺ 453.26/453.2.

Compounds 1019, 1020, 1024, 1023, 1054, 1055, 1056 and 1057 were prepared in a similar fashion, but using the Acylation:EDC method described above.

1019: ¹H NMR (DMSOd6): δ=9.76 (s, 1H, NH), 8.71 (t, 1H, NH), 8.05 (d, 2H, 2CH), 7.94 (d, 2H, 2C_{Ar}H), 7.17 (d, 1H, C_{Ar}H), 6.98 (t, 1H, C_{Ar}H), 6.79 (d, 1H, C_{Ar}H), 6.60 (t, 1H, C_{Ar}H), 4.93 (s, 2H, NH₂), 3.33 (m, 2H, N_{acyl}CH_{2acyclic}), 2.44 (m, 6H, 2NCH_{2cyclic}, NCH_{2acyclic}), 1.70 (m, 6H, 2CH_{2acyclic}, CH_{2cyclic}) ppm. LS-MS (m/z): calcd./found for [M+H]⁺ 367.21/367.2.

1020: ¹H NMR (DMSOd6): δ=9.76 (s, 1H, NH), 8.54 (m, 1H, NH), 8.05 (d, 2H, 2C_{Ar}H), 7.94 (d, 2H, 2C_{Ar}H), 7.17 (d, 1H, C_{Ar}H), 6.97 (d, 1H, C_{Ar}H), 6.78 (d, 1H, C_{Ar}H), 6.59 (d, 1H, C_{Ar}H), 4.93 (s, 2H, NH₂), 3.33 (s, 2H, N_{acyl}CH_{2acyclic}), 2.38 (m, 6H, NCH₂), 1.33 (m, 14H) ppm. LS-MS (m/z): calcd./found for [M+H]⁺ 435.27/435.2.

1023: ¹H NMR (DMSOd6): δ=11.28 (s, 1H, NH), 10.66 (s, 2H, NH₂), 9.05 (s, 1H, C_{6pyr}H), 8.32 (d, 1H, C_{4pyr}H), 8.14 (d, 1H, C_{3pyr}H), 7.65 (d, 1H, C_{Ar}H), 7.52 (d, 1H, C_{Ar}H), 7.37 (m, 2H, 2C_{Ar}H), 3.16 (m, 6H, NCH_{cyclic}, 2CH_{2acyclic}), 1.71 (m, 6H, 3CH_{2cyclic}), 1.36 (m, 6H, 2CH₃) ppm. LS-MS (m/z): calcd./found for [M+H]⁺ 396.24/396.2.

1024: ¹H NMR (DMSOd6): δ=10.72 (s, 1H, NH), 9.09 (s, 1H, C_{6pyr}H), 8.84 (t, 1H, NH), 8.42 (d, 1H, C_{4pyr}H), 8.24 (d, 1H, C_{3pyr}H), 7.92 (d, 2H, C_{Ph}H), 7.37 (t, 2H, C_{Ph}H), 7.13 (t, 1H, C_{Ph}H), 3.52 (m, 1H, NCH_{cyclic}), 3.25 (m, 3H, CH₂, CH), 2.69 (m, 2H, NCH_{2cyclic}), 1.62 (m, 10H, CH_{2cyclic}) ppm. LS-MS (m/z): calcd./found for [M+H]⁺ 379.21/379.2.

1054: ¹H NMR (DMSOd6): δ=10.12 (s, 1H, NH), 9.09 (s, 1H, C_{6pyr}H), 8.82 (d, 1H, NH), 8.40 (m, 1H, C_{4pyr}H), 8.21 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 3.41 (m, 2H, N_{acyl} CH_{2acyclic}), 2.71 (m, 1H, CH_{cyclic}), 2.56 (t, 2H, CH_{2acyclic}), 2.23 (s, 3H, NCH₃), 1.49 (m, 8H, 4CH₂) ppm. LS-MS (m/z): calcd./found for [M+H]⁺ 382.2/380.0.

1055: ¹H NMR (DMSOd6): δ=10.12 (s, 1H, NH), 9.09 (s, 1H, C_{6pyr}H), 8.80 (t, 1H, NH), 8.40 (d, 1H, C_{4pyr}H), 8.22 (d, 1H, C_{3pyr}H), 7.47 (d, 1H, C_{Ar}H), 6.97 (t, 1H, C_{Ar}H), 6.83 (d, 1H, C_{Ar}H), 6.65 (t, 1H, C_{Ad}H), 4.93 (s, 2H, NH₂), 3.37 (m, 2H, N_{acyl}CH_{2acyclic}), 2.58 (m, 2H, NCH_{2acyclic}), 2.51 (m, 4H, 2CH_{2acyclic}), 0.97 (t, 6H, 2CH₃) ppm. LS-MS (m/z): calcd./found for [M+H]⁺ 356.2/356.2.

1056: ¹H NMR (DMSOd6): δ=10.50 (s, 1H, NH), 9.94 (s, 1H, NH), 8.86 (m, 1H, C_{6pyr}H), 8.25 (m, 1H, C_{4pyr}H), 8.09 (d, 1H, C_{3pyr}H), 7.50 (d, 1H, C_{Ar}H), 6.95 (t, 1H, C_{Ar}H), 6.82 (d, 1H, C_{Ar}H), 6.65 (t, 1H, C_{Ar}H), 4.96 (s, 2H, NH₂), 2.13 (s, 3H, CH₃) ppm. LS-MS (m/z): calcd./found for [M+H]⁺ 271.12/271.2.

1057: ¹H NMR (DMSOd6): δ=10.12 (s, 1H, NH), 9.09 (m, 1H, C_{6pyr}H), 8.81 (s, 1H, NH), 8.41 (m, 1H), 8.21 (d, 1H, C_{4pyr}H), 7.47 (d, 1H, C_{Ar}H), 6.97 (t, 1H, C_{Ar}H), 6.83 (d, 1H, C_{Ar}H), 6.65 (t, 1H, C_{Ar}H), 4.93 (s, 2H, NH₂), 3.35 (m, 2H, N_{acyl}CH_{2acyclic}), 2.60 (m, 2H, NCH_{2acyclic}), 2.33 (m, 1H, NCH_{cyclic}), 2.26 (m, 3H, NCH₃), 1.41 (m, 10H, 5CH_{2cyclic}) ppm. LS-MS (m/z): calcd./found for [M+H]⁺ 396.24/396.2.

Synthesis of 1058: Synthesis of 1058 was carried out as shown in the below scheme:

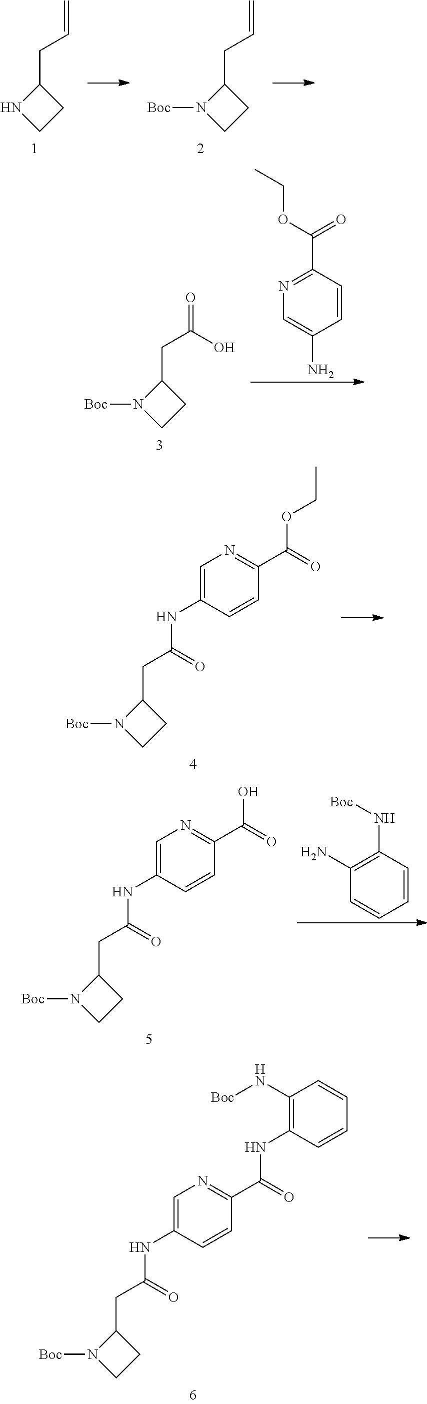

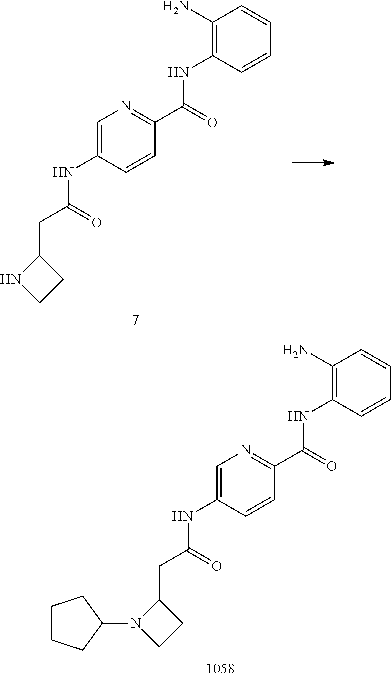 [SMILES: Nc1ccccc1NC(=O)c1ccc(NC(=O)CC2CCN2)cn1 ; Nc1ccccc1NC(=O)c1ccc(NC(=O)CC2CCN2C2CCCC2)cn1]

1058 was synthesized by Boc-deprotection of compound 7 obtained as a result of coupling of 5-(2-(1-(tert-butoxycarbonyl)azetidin-2-yl)acetamido)picolinic acid 5 with mono Boc-protected phenylene diamine 2.

1058: ¹H NMR (400 MHz, Chloroform-d6): δ9.61 (s, 1H), 8.80 (s, 1H), 8.17 (s, 2H), 7.50 (d, J=7.8 Hz, 1H), 7.10 (t, J=7.8 Hz, 1H), 7.02-6.89 (m, 1H), 6.86-6.68 (m, 2H), 6.03 (d, J=15.1 Hz, 1H), 3.92 (s, 1H), 3.77 (d, J=6.8 Hz, 1H), 2.85 (s, 2H), 2.60 (s, 1H), 2.35 (s, 2H), 1.99 (d, J=10.4 Hz, 2H), 1.71 (s, 2H) ppm. LS-MS (m/z): calcd./found for [M+H]⁺ 393.48/393.25.

Synthesis of 1059: To obtain this compound firstly acid 4 was synthesized by the basic hydrolysis of the product 3 which was obtained from acrylamide 2 (see the scheme below). After with the use of general procedure Acylation: EDC followed by Boc-deprotection in the presence of excess of hydrochloric acid the final target was synthesized in accord to the following scheme:

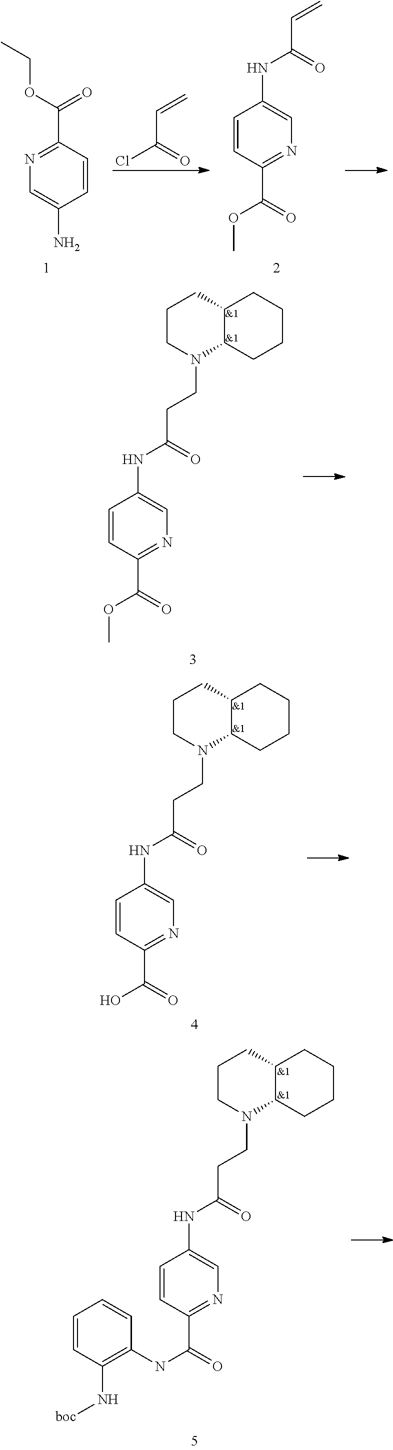

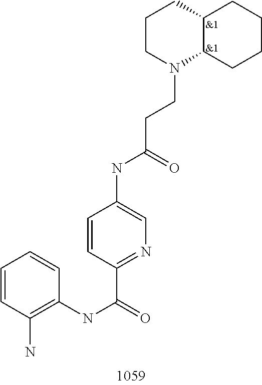 [SMILES: Nc1ccccc1NC(=O)c1ccc(NC(=O)CCN2CCC[C@@H]3CCCC[C@@H]32)cn1]

1059: ¹H NMR (500 MHz, DMSO-d6) δ 11.25 (d, J=8.4 Hz, 1H), 10.64 (s, 1H), 10.56 (d, J=8.0 Hz, 1H), 9.03 (s, 1H), 8.31 (d, J=8.4 Hz, 1H), 8.15 (d, J=8.5 Hz, 1H), 7.63 (d, J=8.0 Hz, 1H), 7.46 (d, J=7.8 Hz, 1H), 7.38 (t, J=7.8 Hz, 1H), 7.32 (t, J=7.6 Hz, 1H), 3.52 (dd, J=13.6, 7.3 Hz, 1H), 3.47-3.39 (m, 1H), 3.32 (dd, J=14.0, 7.0 Hz, 1H), 2.99 (d, J=9.5 Hz, 3H), 2.86 (d, J=10.3 Hz, 1H), 2.27-2.18 (m, 1H), 2.01-1.84 (m, 1H), 1.88-1.74 (m, 2H), 1.73-1.57 (m, 4H), 1.42 (d, J=11.9 Hz, 1H), 1.32-1.12 (m, 3H), 1.07-0.99 (m, 1H) ppm. LS-MS (m/z): calcd./found for [M+H]⁺ 421.5/421.2.

Synthesis of 1060: Synthesis of the compounds 1060 was carried out following the scheme given below:

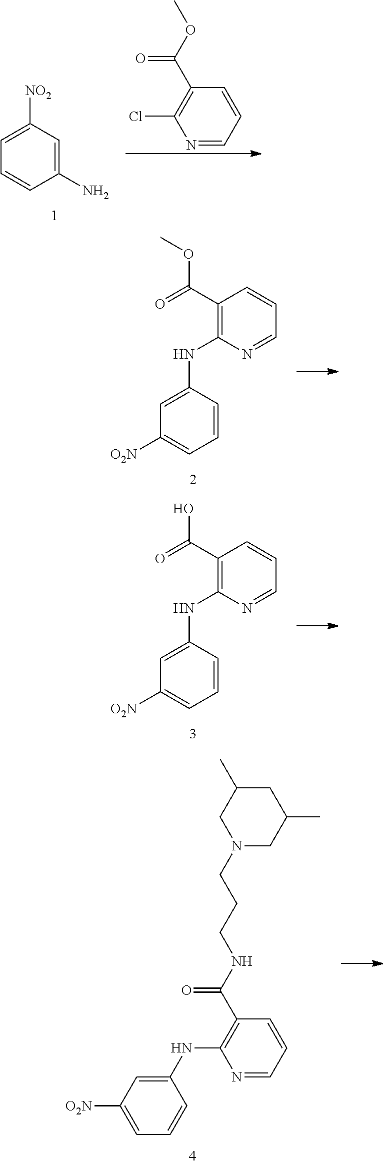 [SMILES: Nc1cccc([N+](=O)[O-])c1 ; COC(=O)c1cccnc1Cl ; COC(=O)c1cccnc1Nc1cccc([N+](=O)[O-])c1 ; O=C(O)c1cccnc1Nc1cccc([N+](=O)[O-])c1 ; CC1CC(C)CN(CCCNC(=O)c2cccnc2Nc2cccc([N+](=O)[O-])c2)C1]

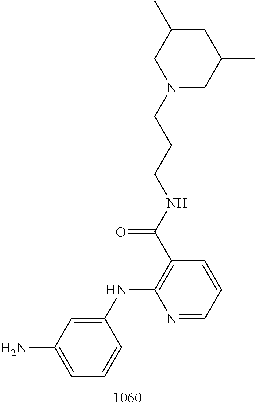 [SMILES: CC1CC(C)CN(CCCNC(=O)c2cccnc2Nc2cccc(N)c2)C1]

1060 was obtained by the coupling of 3-(3,5-dimethylpiperidin-1-yl)propan-1-amine with 2-((3-nitrophenyl)amino)nicotinic acid 3.

1060: ¹H NMR (400 MHz, Chloroform-d6) δ 10.60 (s, 1H), 8.94 (s, 1H), 8.27 (d, J=4.7 Hz, 1H), 7.70 (d, J=7.7 Hz, 1H), 7.12-6.89 (m, 2H), 6.60 (dd, J=7.6, 4.9 Hz, 1H), 6.32 (d, J=7.7 Hz, 1H), 3.51 (q, J=5.3 Hz, 2H), 3.03-2.81 (m, 2H), 2.53 (t, J=5.4 Hz, 2H), 1.84-1.70 (m, 3H), 1.43 (t, J=11.1 Hz, 2H), 0.84 (d, J=6.5 Hz, 6H), 0.55 (q, J=12.1 Hz, 1H) ppm. LS-MS (m/z): calcd./found for [M+H]⁺ 381.5/381.2.

Synthesis of 1061: To obtain this compound firstly acid 3 was synthesized by the coupling of 6-((2-nitrophenyl)amino)nicotinic acid with (1-cyclopentylazetidin-2-yl)methanamine (see the scheme below).

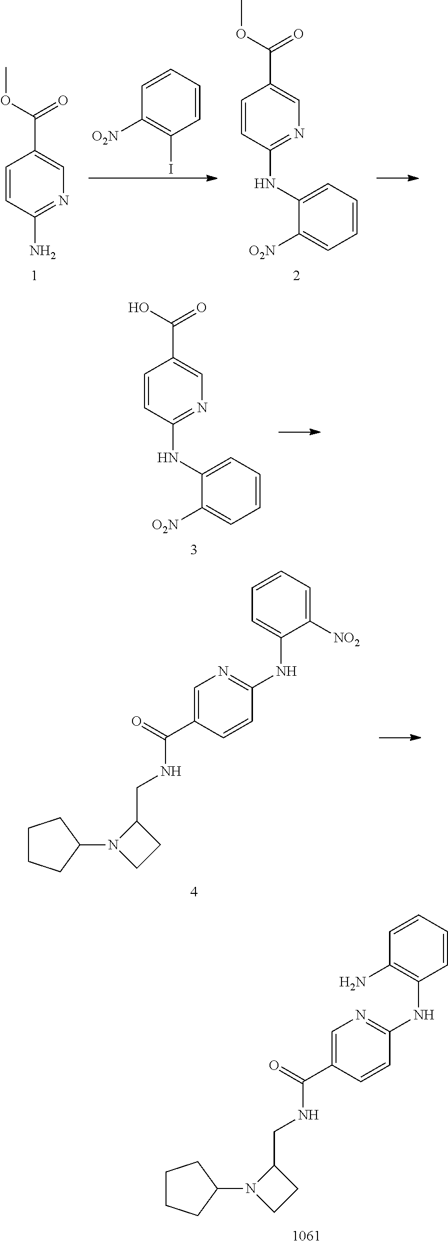

1061: LS-MS (m/z): calcd./found for [M+H]+ 365.26/365.5.

Synthesis of 1062: Synthesis of the compounds 1062 was carried out following the scheme given below:

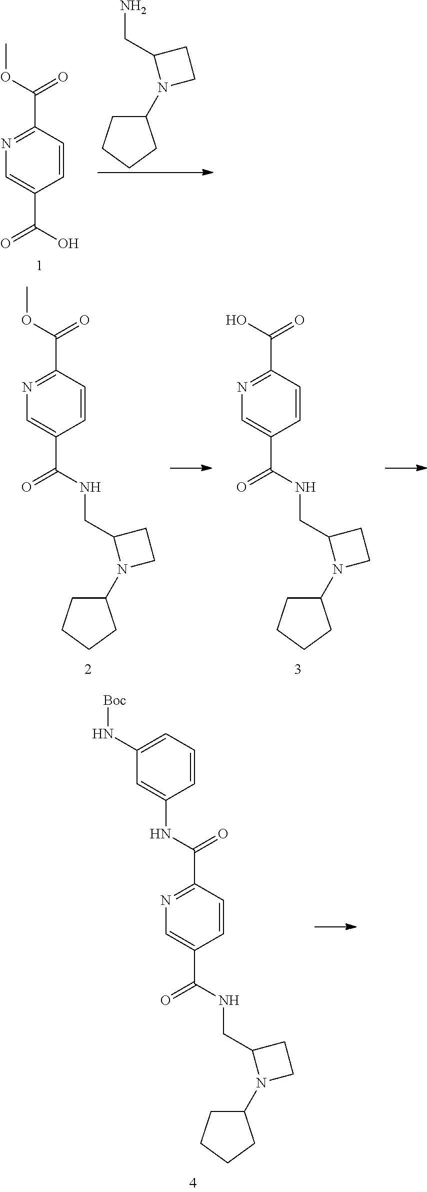

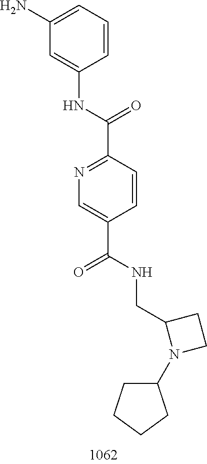 [SMILES: Nc1cccc(NC(=O)c2ccc(C(=O)NCC3CCN3C3CCCC3)cn2)c1]

1062: LS-MS (m/z): calcd./found for [M+H]+ 393.25/393.48.

Synthesis of 1063: Synthesis of the compound 1063 was carried out following the scheme given below.

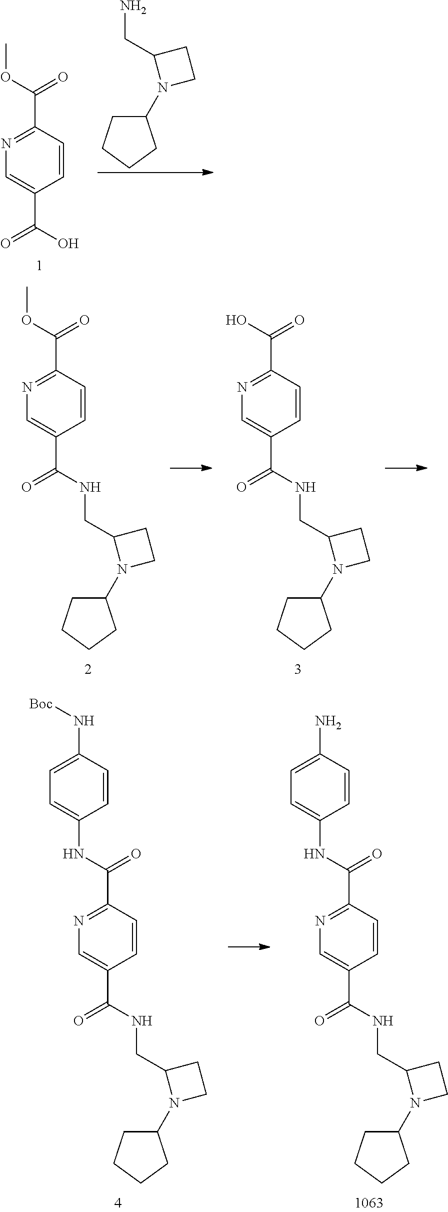

1062: ¹H NMR (400 MHz, Chloroform-d6) δ 9.80 (s, 1H), 9.03 (s, 1H), 8.47-8.16 (m, 2H), 7.55 (d, J=8.2 Hz, 3H), 6.70 (d, J=8.3 Hz, 2H), 3.72 (dd, J=14.5, 7.5 Hz, 1H), 3.63 (s, 2H), 3.52 (s, 1H), 3.40-3.17 (m, 2H), 2.95-2.70 (m, 2H), 1.96 (dt, J=18.2, 8.5 Hz, 3H), 1.65 (q, J=8.1, 6.4 Hz, 4H), 1.34 (d, J=15.2 Hz, 2H) ppm. LS-MS (m/z): calcd./found for [M+H]+ 393.25/393.48.

Synthesis of 1064: To obtain this compound firstly acid 3 was synthesized by the basic hydrolysis of the product of coupling of 6-(methoxycarbonyl)nicotinic acid 1 with tert-butyl (2-aminophenyl)carbamate (see the scheme below).

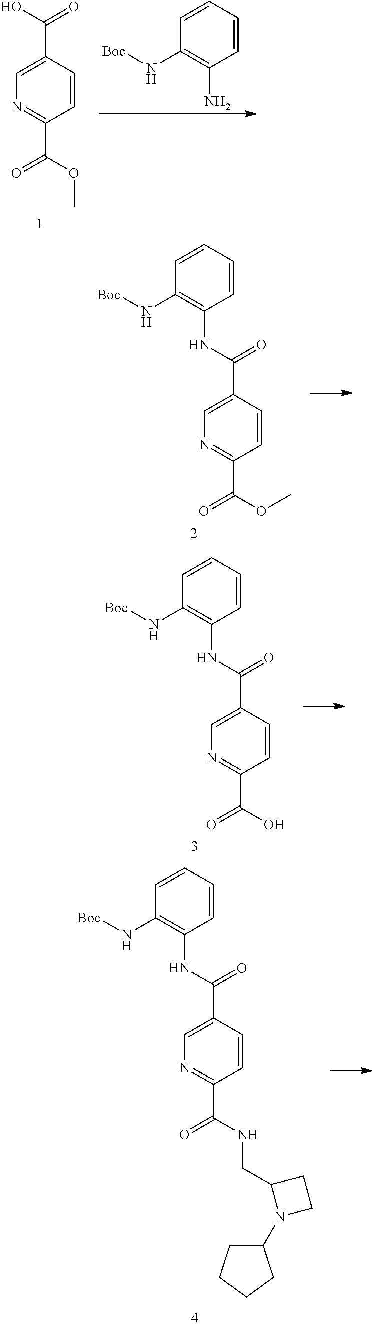 [SMILES: COC(=O)c1ccc(C(=O)O)cn1 ; CC(C)(C)OC(=O)Nc1ccccc1N ; COC(=O)c1ccc(C(=O)Nc2ccccc2NC(=O)OC(C)(C)C)cn1 ; CC(C)(C)OC(=O)Nc1ccccc1NC(=O)c1ccc(C(=O)O)nc1 ; CC(C)(C)OC(=O)Nc1ccccc1NC(=O)c1ccc(C(=O)NCC2CCN2C2CCCC2)nc1]

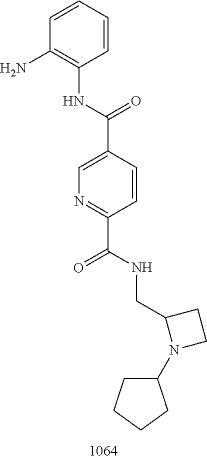 [SMILES: Nc1ccccc1NC(=O)c1ccc(C(=O)NCC2CCN2C2CCCC2)nc1]

1064: ¹H NMR (400 MHz, Chloroform-d) δ 9.08 (s, 1H), 8.58 (s, 1H), 8.39-8.12 (m, 3H), 7.38 (d, J=7.9 Hz, 1H), 7.09 (t, J=7.7 Hz, 1H), 6.84 (d, J=7.7 Hz, 2H), 3.87 (s, 2H), 3.76-3.61 (m, 1H), 3.43-3.22 (m, 3H), 2.79 (dt, J=34.1, 7.3 Hz, 2H), 1.93 (dd, J=20.4, 11.3 Hz, 2H), 1.49 (s, 2H), 1.35 (s, 2H) ppm. LS-MS (m/z): calcd./found for [M+H]+ 393.25/393.48.

Synthesis of the compounds 1065, 1066, 1067, 1068, 1069: To obtain compounds noted, acid 3 was synthesized in accordance with the following the scheme to provide the compound:

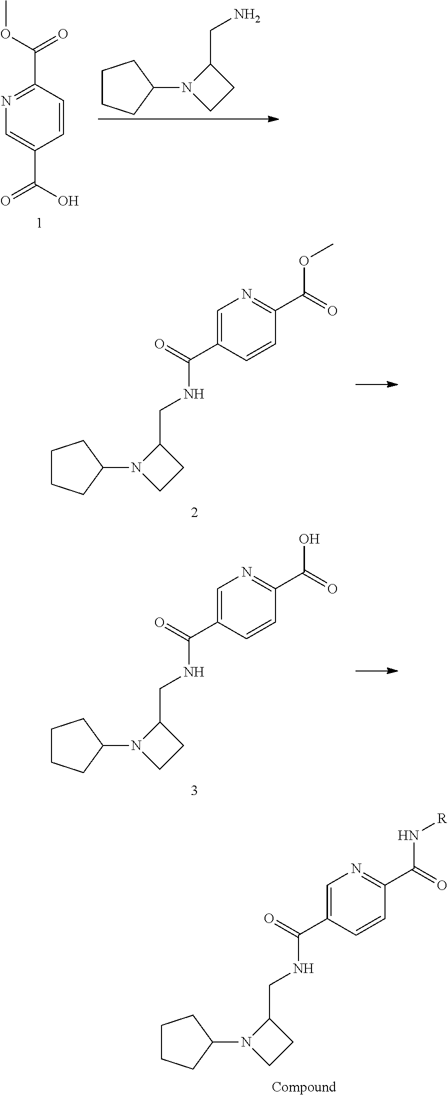

After with the use of general procedure Acylation: HATU the final compounds were synthesized.

1065: LS-MS (m/z): calcd./found for [M+H]+ 396.2/396.48.

1066: LS-MS (m/z): calcd./found for [M+H]+ 421.3/421.54.

1067: LS-MS (m/z): calcd./found for [M+H]+ 446.23/446.46.

1068: LS-MS (m/z): calcd./found for [M+H]+ 471.31/471.59.

1069: LS-MS (m/z): calcd./found for [M+H]+ 411.47/411.24.

Synthesis of the 1070 was carried out following the scheme given below:

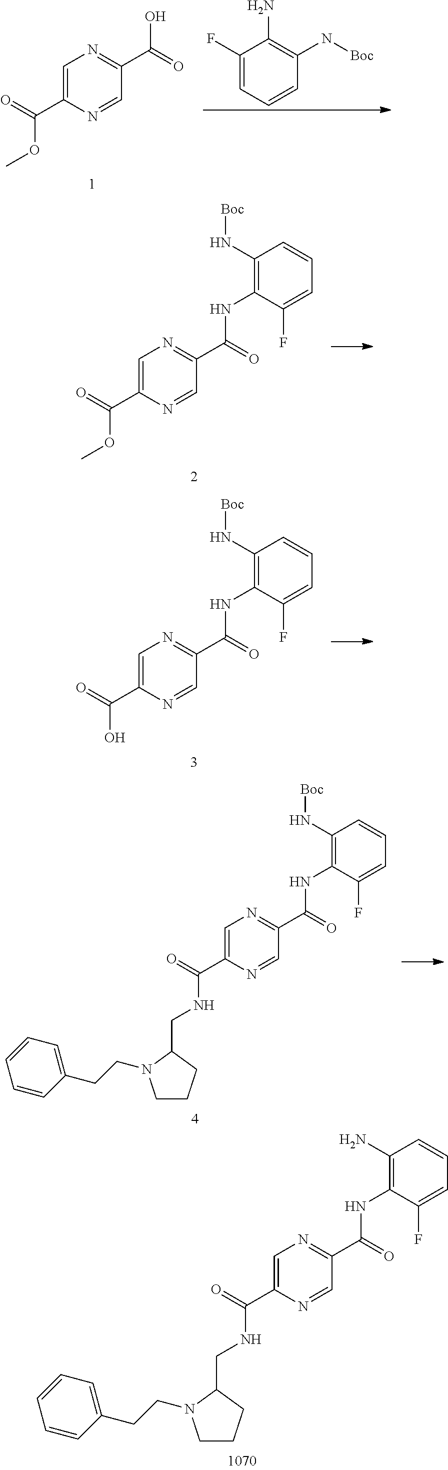

1070: LS-MS (m/z): calcd./found for [M+H]+ 462.25/462.52.

### Biological Assays

CI-994 was purchased from Selleckchem, and niclosamide was purchased from Sigma. The Super8XTOPFlash luciferase construct was provided by Dr. Randall T. Moon (University of Washington, Seattle, Wash.). The β-galactosidase-expressing vector was obtained from Promega. Plasmid pcDNA3-Wnt3 Å-HA was constructed as described previously [32]. Rabbit monoclonal anti-BACE1 (#5606), anti-phospho-Tau (Ser202) (#39357), anti-LRP6 (#2560), anti-GSK3β(27C10) (#9315), anti-phospho-GSK3β(D3A4) (#9322) were purchased from Cell Signaling Technology. Mouse monoclonal anti-β-catenin (#61054) was from BD Biosciences. Mouse monoclonal anti-tau (#MN1000B, HT7) was from Thermo Fisher Scientific. Mouse monoclonal anti-actin (#A2228) was from Sigma. Peroxidase labeled anti-mouse and anti-rabbit secondary antibodies and ECL system were purchased from Amersham Life Science. IRDye® 680RD goat anti-rabbit IgG (H+L) and IRDye® 680RD goat anti-mouse IgG (H+L) were from Li-Cor Corporation. The luciferase and β-galactosidase assay systems were from Promega. Fetal bovine serum (FBS) was purchased from Sigma. Tissue culture media, fetal bovine serum (FBS), and plastic-ware were obtained from Life Technologies, Inc. Proteinase inhibitor cocktail Complete™ was obtained from Boehringer Mannheim.

Cell culture: Human fibrosarcoma cancer HT1080 oells stably transfected with HA-tagged LRP6 have been described before [33]. HEK293 cells, C2C12 cells and N2a cells were obtained from American Type Culture Collection. HEK293 cells, C2C12 cells, N2a cells and LRP6-HT1080 cells were cultured in Dulbeccos minimum essential medium containing 10% fetal bovine serum, 2 mM L-glutamine, 100 units/ml penicillin and 100 μg/ml streptomycin. Human blood-brain barrier hCMEC/D3 cells was purchased from EMD Millipore, and were cultured in EndoGRO™ medium (Lonza) supplemented with 1 ng/ml FGF2 (#GF003) from EMD Millipore. All the cells were maintained at 37° C. in humidified air containing 5% CO₂.

Luciferase reporter assay for Wnt/β-catenin signaling: HEK293 cells were plated into 24-well plates. After overnight culture, the cells were transiently transfected with the Super8XTOPFlash luciferase construct and β-galactosidase-expressing vector along with or without Wnt3A plasmid. After 24 h incubation, cells were treated with each individual compound at the indicated concentrations for 24 h. Cells were then lysed and both luciferase and β-galactosidase activities were determined. The luciferase activity was normalized to the β-galactosidase activity.

Western blotting: Cells in 6-well plates were lysed in 0.5 ml of lysis buffer (phosphate-buffered saline containing 1% Triton X-100, 1× Proteinase inhibitor cocktail Complete, and 1 mM PMSF) at 4° C. for 15 min. Equal quantities of protein were subjected to SDS-PAGE under reducing conditions. Following transfer to immobilon-P transfer membrane, successive incubations with a primary antibody, and a horseradish peroxidase-conjugated secondary antibody or IRDye® 680RD goat anti-rabbit/mouse secondary antibodies were carried out for 60-120 min at room temperature. The immunoreactive proteins were then detected using the ECL system or the Odyssey infrared imaging system (LI-COR). Films showing immunoreactive bands were scanned by Hp Scanjet 5590.

Alkaline phosphatase (ALP) activity assay: C2C12 cells in 24-well plates were treated with each individual compound at the indicated concentrations for 72 h. Cells were then harvested for assay of ALP activity with the ALP assay kit (AnaSpec) according to the manufacturers specifications.

Histone Deacetylase (HDAC) activity assay: Histone Deacetylase (HDAC) activity assay kit (ab156064) was purchased from Abcam, and the effects of the tested compounds on HDAC activity were examined according to the manufacturers specifications. Results are shown in FIG. 12.

Cell viability assay: Human blood-brain barrier hCMEC/D3 cells were seeded into 96-well tissue culture treated microtiter plates at a density of 20,000 cells/well. After 24 h incubation, the cells were treated with the test compounds at the indicated concentrations for 24 h. Cell viability was measured by the CellTiter-Glo Assay (Promega), which is a luminescent assay that is an indicator of live cells as a function of metabolic activity and ATP content.

Aqueous solubility assay: Using a 10 mM stock solution of the compound in 100% DMSO, dilutions were prepared to a theoretical concentration of 200 μM in duplicates in phosphate-buffered saline pH 7.4 with 2% final DMSO. In parallel, compound dilutions in 50% acetonitrile/PBS were prepared to theoretical concentrations of 0 μM (blank), 50 μM, 100 μM and 200 μM with 2% final DMSO to generate calibration curves. The experimental compound dilutions in PBS were further allowed to equilibrate at 25° C. on a thermostatic orbital shaker for two hours and then centrifuged at 14000 rpm, 5 min and filtered through HTS filter plates using a vacuum manifold. The supernatants of test compounds were diluted 2-fold with acetonitrile contained 4% DMSO before measuring. Ondansetron was used as reference compound to control proper assay performance. 200 μl of samples were transferred to 96-well plate and measured in 200-550 nm range with 5 nm step. The effective range of this assay is approximately 2-200 μM and the compounds returning values close to the upper limit of the range may have higher actual solubility (e.g. 5 [symbol] deoxy-5-fluorouddine). Results are shown in the following Table.

TABLE

| Compound | PBS solubility, pH 7.4, μM | | | |
|---|---|---|---|---|
| ID | Exp1 | Exp2 | Mean | SE |
| Ondansetron | 109 | 113 | 111 | 2 |
| (as control/ | | | | |
| reference) | | | | |
| 1009 | 197 | 191 | 194 | 3 |
| 1011 | 207 | 207 | ≥200 | — |
| 1015 | 210 | 211 | ≥200 | — |
| 1020 | 200 | 205 | ≥200 | — |

Metabolic stability in mouse liver microsomes: Mouse hepatic microsomes were isolated from pooled, perfused livers of Balb/c male mice according to the standard protocol. The batch of microsomes was tested for quality control using Imipramine, Propranolol and Verapamil as reference compounds. Microsomal incubations were carried out in 96-well plates in 5 aliquots of 40 μL each (one for each time point). Liver microsomal incubation medium contained PBS (100 mM, pH 7.4), MgCl₂ (3.3 mM), NADPH (3 mM), glucose-6-phosphate (5.3 mM), glucose-6-phosphate dehydrogenase (0.67 units/ml) with 0.42 mg of liver microsomal protein per ml. Control incubations were performed replacing the NADPH-cofactor system with PBS. Test compound (2 μM, final solvent concentration 1.6%) was incubated with microsomes at 37° C., shaking at 100 rpm. Incubations were performed in duplicates. Five time points over 40 minutes had been analyzed. The reactions were stopped by adding 12 volumes of 90% acetonitrile-water to incubation aliquots, followed by protein sedimentation by centrifuging at 5500 rpm for 3 minutes. Incubations were performed in duplicates. Supernatants were analyzed using the HPLC system coupled with tandem mass spectrometer. Results are shown in FIG. 14.

The elimination constant (kₑₗ), half-life (t_{1/2}) and intrinsic clearance (Clᵢₙₜ) were determined in plot of In(AUC) versus time, using linear regression analysis:

$${k}_{el} = - slope \\ {t}_{1 / 2} = \frac{0.693}{k} \\ {Cl}_{int} = \frac{0.693}{{t}_{1 / 2}} \times \frac{μ {l}_{incubation}}{{mg}_{microsomes}}$$

Ames test: According to the OECD Guidelines For Testing Of Chemicals, experiments with the tested agents, positive and negative controls, were conducted in triplicates on 6 tester strains, S. typhimurium: TA98, TA100, TA1535, TA1537 and E. coli: wp2[pKM101]+wp2 uvrA mixed 1:2. Dimethyl sulfoxide (DMSO) was used as the vehicle control for all tester strains in mutagenicity assay with and without metabolic activation. Compounds with known mutagenic activity were used as the positive controls in mutagenicity assay without metabolic activation: 2-nitrofluorene (0.1 μg/plate) for TA98, 4-nitroquinoline N-oxide (0.1 μg/plate) for TA100, sodium azide (0.2 μg/plate) for TA1535, 9-aminoacridine (7.5 μg/plate) for TA1537, and 4-nitroquinoline N-oxide (0.1 μg/plate) for E. coli. 2-Aminoantracene in final concentration 0.4 μg/ml was used as the positive control (the solution of 2-aminoantracene in DMSO was prepared immediately before experiment) in test with metabolic activation. For each experiment tester strain cultures were grown overnight in Oxoid nutrient broth #2 at 37° C. with shaking at 235 rpm to a density of 1-2×10⁹ colony forming units/ml (OD₆₀₀˜1.5). The S9 mix, which is consist of 9000 g supernatant fraction of rat liver homogenate (S9 fraction), was prepared immediately prior to its use in experimental procedure.

The tester strain (10 μl of overnight culture) was mixed with the test agent (10 μl of the DMSO stock) and GM liquid medium (30 μl) or S9 mix (30 μl). Then, 200 μl of molten top agar supplemented with histidine/biotin (final concentration 0.05 mM) for S. typhimurium and tryptophan (final concentration 0.025 mM) for E. coli were added to the tube (top agar was kept at 44° C. to prevent hardening). The mixed suspension was poured onto the surface of a GM agar plate (2.0 ml of GM agar per plate). After the top agar solidified, the plates were inverted and incubated at 37° C. for 48 hours. The results were expressed as number of revertant colonies per plate. The final concentrations of the test compounds in the mutagenicity assay with all tester strains in both the presence and absence of S9 mix were 400 μM. All concentrations were tested in triplicates.

In addition, bacterial cytotoxicity evaluation was performed using E. coli strain TOP10-pTac-lux-CDABE, expressing both luciferase (a heterodimer of LuxA and LuxB subunits) and the enzymes required for the production of luciferase substrate (LuxC, LuxD and LuxE). For the experiment, bacterial culture was grown overnight in LB medium at 37° C. with shaking at 250 rpm. The overnight culture was diluted 1:666 into fresh LB medium supplemented with 50 μg/ml kanamycin and incubated at 37° C. and 250 rpm to OD600 0.4-0.5. Serial dilutions of the test compounds in DMSO were prepared (40 mM, 20 mM, 10 mM, 5 mM, 2.5 mM, 1.25 mM, 0.625 mM) and 4 μl aliquots of each solution were transferred into 96-well plate and mixed with 96 μl of 0.9% NaCl. Then, 10 μl of the dilutions were dispensed into the appropriate wells of 384-well white screening microplate. 30 μl aliquots of freshly grown bacterial culture at OD600 of 0.4-0.5 were added to each well with compounds' dilutions (Final concentration of the test compounds were 400 μM, 200 μM, 100 μM, 50 μM, 25 μM, 12.5 μM, 6.25 μM). The plate was incubated for 4 h at 37° C. and the luminescence. Results are shown in the below tables.

TABLE
Bacterial reverse mutation assays (without metabolic activation)

| | Bacterial strain | | | | |
|---|---|---|---|---|---|
| Compound | TA 98 | TA100 | TA1535 | TA1537 | E. coli |
| Negative control^{a} | 1.3 ± 1.2^{d} | 6.0 ± 1.7 | 1.7 ± 0.6 | 0.3 ± 0.6 | 7.7 ± 2.9 |
| Positive control^{b} | 26.3 ± 4.5 | 389.1 ± 104.4 | 133.7 ± 9.9 | 130.3 ± 27.0 | 45.0 ± 12.78 |
| 1011 | 1.7 ± 1.5 | 4.3 ± 3.5 | 1.7 ± 2.1 | 0.7 ± 0.6 | 8.7 ± 2.1 |
| 1015 | 1.3 ± 1.5 | 1.33 ± 0.6 | 0.3 ± 0.6 | 0.0 ± 0.0 | 5.3 ± 4.7 |

TABLE
Bacterial reverse mutation assays (with metabolic activation)

| | Bacterial strain | | | | |
|---|---|---|---|---|---|
| Compound | TA 98 | TA100 | TA1535 | TA1537 | E. coli |
| Negative control^{a} | 1.3 ± 0.6 ^{d} | 5.3 ± 2.5 | 1.0 ± 1.0 | 0.33 ± 0.6 | 10.7 ± 1.2 |
| Positive control^{b} | 53.7 ± 9.3 | 122.0 ± 10.8 | 11.3 ± 12.1 | 5.33 ± 0.6 | 25.0 ± 1.7 |
| 1011 | 1.7 ± 2.1 | 4.7 ± 2.1 | 0.7 ± 0.6 | 0.0 ± 0.0 | 9.7 ± 3.2 |
| 1015 | 0.7 ± 1.2 | 2.0 ± 1.0 | 0.7 ± 1.2 | 0.0 ± 0.0 | 9.0 ± 2.7 |

^{a}Vehicle (DMSO)
^{b}2-nitrofluorene (0.1 μg/plate) for TA98, 4-nitroquinoline N-oxide (0.1 μg/plate) for TA100 and E. coli, sodium azide (0.2 μg/plate) for TA1535, and 9-aminoacridine (7.5 μg/plate) for TA1537.
^{c}1011 and 1015 were tested at 400 μM
^{d} Number of revertant colonies per plate, mean ± SD, n = 3

Pharmacokinetic study of compound 1015 in Balb/c mice following intravenous administration: Male Balb/c mice (11-12 weeks old, body weight 21.9 to 31.3 g and average body weight across all groups 25.6 g, SD=2.19 g) were used in this study. The animals were randomly assigned to the treatment groups before the pharmacokinetic study; all animals were fasted for 4 h before intravenous injection of 1015 at 2 mg/kg dissolved in Kolliphor HS-physiological saline (20%:80%). Eight time points (5, 15, 30, 60, 120, 240, 480 and 1440 min) were set for this pharmacokinetic study. Each of the time point treatment group included 4 animals. There was also a control group of 2 animals. Mice were injected IV with 2,2,2-tribromoethanol at the dose of 150 mg/kg prior to drawing the blood. Blood collection was performed from the orbital sinus in microtainers containing K2EDTA. Animals were sacrificed by cervical dislocation after blood samples collection. Brain and plasma samples were immediately processed, flash-frozen and stored at −70° C. until subsequent analysis.

Plasma samples (50 μl) were mixed with 200 μl of IS solution. After mixing by pipetting and centrifuging for 4 min at 6000 rpm, 2 μl of each supernatant was injected into LC-MS/MS system. Solution of compound IS-22665-01 (200 ng/ml in acetonitrile-methanol mixture, 1:1, v/v) was used as internal standard (IS) for quantification of 1015 in plasma samples. Brain samples (100 mg±1 mg) were dispersed in 500 μl of IS200(80) using zirconium oxide beads (150 mg±5 mg) in The Bullet Blender® homogenizer for 60 seconds at speed 8. After dispersing and centrifuging for 4 min at 14000 rpm, 2 μl of each supernatant was injected into LC-MS/MS system. Solution of compound IS-22665-01 (200 ng/ml in water-acetonitrile-methanol mixture, 2:4:4, v/v/v) was used as internal standard (IS200(80)) for quantification of 1015 in brain samples. The pharmacokinetic data analysis was performed using noncompartmental, bolus injection or extravascular input analysis models in WinNonlin 5.2 (PharSight).

HEK293 cells were plated into 24-well plates. After overnight culture, the cells were transiently transfected with the Super8XTOPFlash luciferase construct and β-galactosidase-expressing vector along with the Wnt3A plasmid. After 24 h incubation, the cells were treated with each individual compound at 30-30,000 nM in triplication for 24 h. The cells were then lysed, and luciferase and β-galactosidase activities were determined with the luciferase assay system (Promega, Cat. #E1501) and β-galactosidase assay system (Promega, Cat. #E2000), respectively. The luciferase activity was normalized to the s-galactosidase activity, and the EC₅₀ values were calculated by use of GraphPad software, and shown in the below table. All the EC₅₀ values (mean and SD) are from 3-7 independent experiments.

| Compound | EC₅₀ ± SD (nM) |
|---|---|
| 1009 | 601 ± 101 |
| 1011 | 920 ± 455 |
| 1013 | 444 ± 36 |
| 1015 | 623 ± 212 |
| 1020 | 860 ± 278 |
| 1045 | 507 ± 128 |
| 1046 | 532 ± 38 |
| 1050 | 408 ± 77 |
| 1023 | 496 ± 195 |
| 1038 | 526 ± 268 |
| 1039 | 298 ± 25 |
| 1041 | 596 ± 79 |
| 1021 | 1385 ± 643 |
| 1056 | 1610 ± 197 |
| 1028 | 519 ± 24 |
| 1030 | 483 ± 40 |
| 1059 | 494 ± 147 |

1015 activates Wnt/β-catenin signaling in iPSC-derived human neurons and suppresses tau phosphorylation: Neurons derived from patient-specific induced pluripotent stem cells (iPSCs) can recapitulate the major cellular phenotypes of AD and hence can serve as excellent disease models for drug development 1015 enhances Wnt co-receptor LRP6 level (FIG. 16 at A) and increases Wnt specific target axin2 expression in human iPSC-derived neurons (FIG. 16 at B), indicating that 1015 is able to activate Wnt/β-catenin signaling in human neurons. NeuroD1, a proneural basic helix-loop-helix (bHLH) transcription factor, plays a critical role in CNS development, and is important for survival of neuronal progenitor cells. There is a TCF/LEF regulatory element in the Neurod1 promoter, and NeuroD1 is a specific target of Wnt/β-catenin signaling. 1015 significantly enhances NeuroD1 expression in human iPSC-derived neurons (FIG. 16 at C). In addition, 1015 also induces neural stem cell marker SOX2 expression in human iPSC-derived neurons (FIG. 16 at D). Importantly, 1015 suppresses tau phosphorylation in human iPSC-derived neurons (FIG. 16 at D).

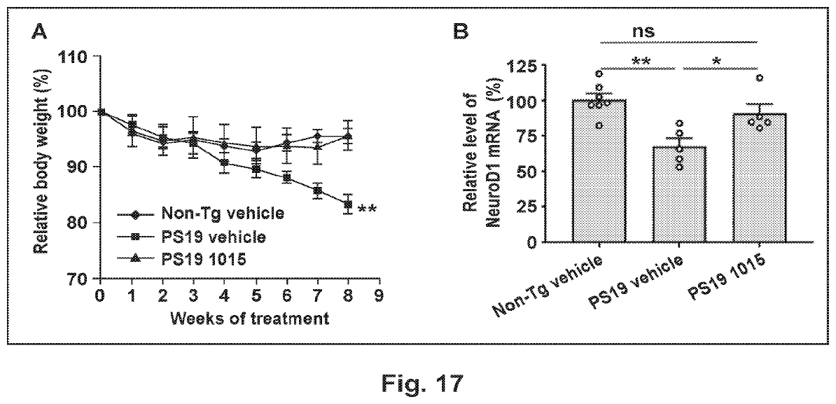

1015 activates Wnt/β-catenin signaling and prevents body weight loss in PS19 mice: PS19 is a widely used tauopathy model which recapitulates many AD-related phenotypes. PS19 mice displays neuronal loss and brain atrophy by eight months, and develops neurofibrillary tangles (NTFs) at six months with progressive accumulation. Therefore, the PS19 mouse model was used for efficacy studies. There was a noticeable drop in weight in all groups after initiation of daily dosing in each group, which is most likely a consequence of the sudden daily manipulation of the mice (FIG. 17 at A). Importantly, 1015 significantly prevented body weight loss over the course of the 8-week treatment in PS19 mice (FIG. 17 at A). Indeed, 1015 restored, but not over induced, neuroD1 expression in brain of PS-19 mice (FIG. 17 at B), indicating that 1015 is able to restore, but not overactivate, Wnt/β-catenin signaling in brain of PS19 mice.

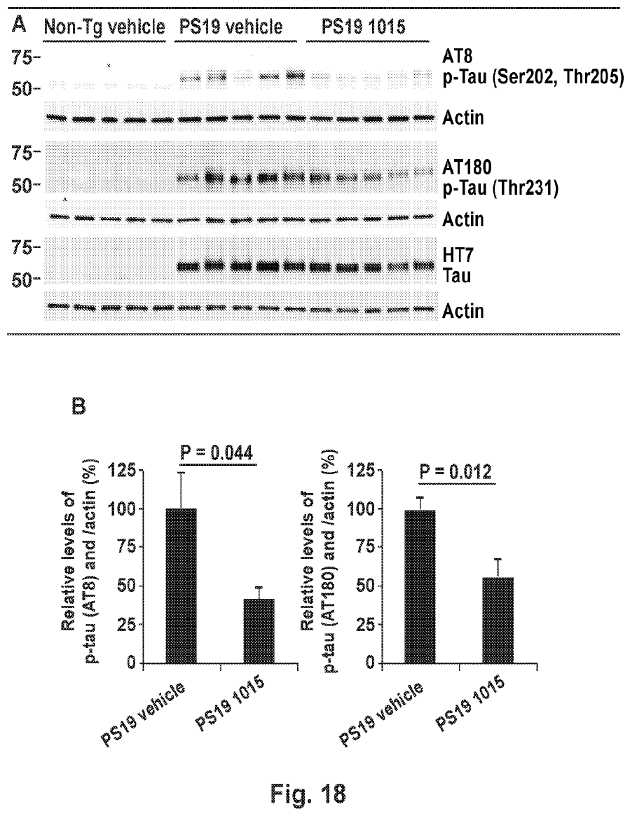

1015 suppresses tau phosphorylation in PS19 mice: One of the two major hallmarks of AD is the presence of NFTs, which are composed of hyperphosphorylated forms of tau protein in neurons. Increased phosphorylation of tau and subsequent microtubule instability and transport defects are responsible to neuronal death. It was found that 1015 significantly suppresses tau phosphorylation in cortex of PS-19 mice (FIG. 18).

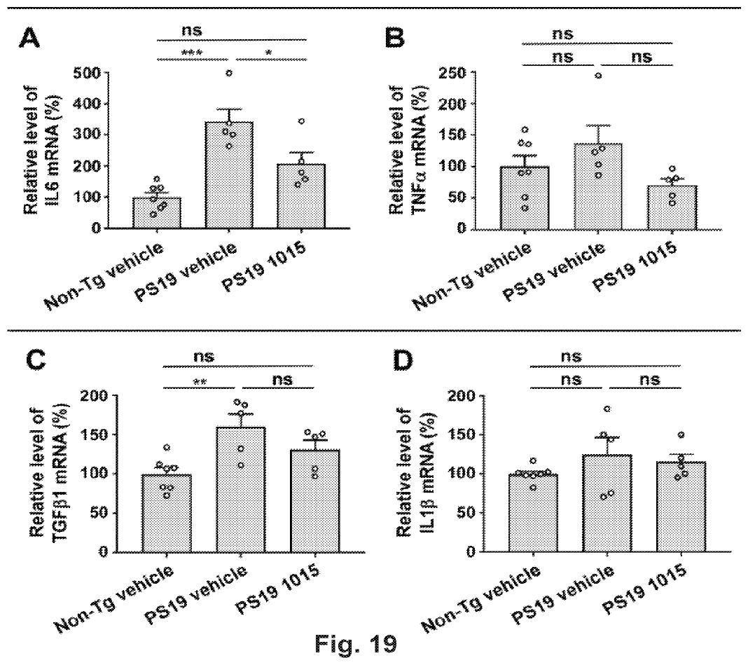

1015 suppresses neuroinflammation in PS19 mice: Glia-mediated neuroinflammation is another pathological hallmark of AD. Activation of Wnt/β-catenin signaling suppresses neuroinflammation in mouse AD models. Compared to non-Tg mice, PS19 mice displayed higher levels of neuroinflammatory cytokines IL-6, TNFα, TGFβ1 and IL1s (FIG. 19). Importantly, 1015 treatment inhibited neuroinflammatory cytokine expression, with more profound inhibition found in IL6 expression (FIG. 19), suggesting that 1015 can inhibit neuroinflammation in PS19 mice.

### REFERENCES

[1] C. Niehrs, The complex world of WNT receptor signalling, Nat Rev Mol Cell Biol, 13 (2012) 767-779.
[2] R. Nusse, H. Clevers, Wnt/beta-Catenin Signaling, Disease, and Emerging Therapeutic Modalities, Cell, 169 (2017) 985-999.
[3] D. J. Selkoe, J. Hardy, The amyloid hypothesis of Alzheimers disease at 25 years, EMBO Mol Med, 8 (2016) 595-608.
[4] Y. Mu, F. H. Gage, Adult hippocampal neurogenesis and its role in Alzheimers disease, Mol Neurodegener, 6 (2011) 85.
[5] R. D. Brinton, J. M. Wang, Therapeutic potential of neurogenesis for prevention and recovery from Alzheimers disease: allopregnanolone as a proof of concept neurogenic agent, Curr Alzheimer Res, 3 (2006) 185-190.
[6] N. C. Inestrosa, L. Varela-Nallar, Wnt signaling in the nervous system and in Alzheimers disease, J Mol Cell Biol, 6 (2014) 64-74.
[7] C. C. Liu, C. W. Tsai, F. Deak, J. Rogers, M. Penuliar, Y. M. Sung, J. N. Maher, Y. Fu, X. Li, H. Xu, S. Estus, H. S. Hoe, J. D. Fryer, T. Kanekiyo, G. Bu, Deficiency in LRP6-mediated Wnt signaling contributes to synaptic abnormalities and amyloid pathology in Alzheimers disease, Neuron, 84 (2014) 63-77.
[8] G. V. De Ferrari, A. Papassotiropoulos, T. Biechele, F. Wavrant De-Vrieze, M. E. Avila, M. B. Major, A. Myers, K. Saez, J. P. Henriquez, A. Zhao, M A. Wollmer, R. M. Nitsch, C. Hock, C. M. Morris, J. Hardy, R. T. Moon, Common genetic variation within the low-density lipoprotein receptor-related protein 6 and late-onset Alzheimers disease, Proc Natl Acad Sci USA, 104 (2007) 9434-9439.
[9] M. A. Alarcon, M. A. Medina, Q. Hu, M. E. Avila, B. I. Bustos, E. Perez-Palma, A. Peralta, P. Salazar, G. D. Ugarte, A. E. Reyes, G. M. Martin, C. Opazo, R. T. Moon, G. V. De Ferrari, A novel functional low-density lipoprotein receptor-related protein 6 gene alternative splice variant is associated with Alzheimers disease, Neurobiol Aging, 34 (2013) 1709 e1709-1718.
[10] C. Parr, N. Mirzaei, M. Christian, M. Sastre, Activation of the Wnt/beta-catenin pathway represses the transcription of the beta-amyloid precursor protein cleaving enzyme (BACE1) via binding of T-cell factor-4 to BACE1 promoter, FASEB J, 29 (2015) 623-635.
[11] C. Tapia-Rojas, P. V. Burgos, N.C. Inestrosa, Inhibition of Wnt signaling induces amyloidogenic processing of amyloid precursor protein and the production and aggregation of Amyloid-beta (Abeta)42 peptides, J Neurochem, 139 (2016) 1175-1191.
[12] C. Tapia-Rojas, N. C. Inestrosa, Wnt signaling loss accelerates the appearance of neuropathological hallmarks of Alzheimers disease in J20-APP transgenic and wild-type mice, J Neurochem, 144 (2018) 443-465.
[13] C. Scali, F. Caraci, M. Gianfriddo, E. Diodato, R. Roncarati, G. Pollio, G. Gaviraghi, A. Copani, F. Nicoletti, G. C. Terstappen, A. Caricasole, Inhibition of Wnt signaling, modulation of Tau phosphorylation and induction of neuronal cell death by DKK1, Neurobiol Dis, 24 (2006) 254-265.
[14] A. Caricasole, A. Copani, F. Caraci, E. Aronica, A. J. Rozemuller, A. Caruso, M. Storto, G. Gaviraghi, G. C. Terstappen, F. Nicoletti, Induction of Dickkopf-1, a negative modulator of the Wnt pathway, is associated with neuronal degeneration in Alzheimers brain, J Neurosci, 24 (2004) 6021-6027.
[15] G. Esposito, D. De Filippis, R. Camuccio, A. A. Izzo, T. luvone, The marijuana component cannabidiol inhibits beta-amyloid-induced tau protein hyperphosphorylation through Wnt/beta-catenin pathway rescue in PC12 cells, J Mol Med (Ber), 84 (2006) 253-258.
[16] N. C. Inestrosa, E. Arenas, Emerging roles of Wnts in the adult nervous system, Nat Rev Neurosci, 11 (2010) 77-86.
[17] V. Budnik, P. C. Salinas, Wnt signaling during synaptic development and plasticity, Curr Opin Neurobiol, 21 (2011) 151-159.
[18] R. A. Quintanilla, F. J. Munoz, M. J. Metcalfe, M. Hitschfeld, G. Olivares, J. A. Godoy, N. C. Inestrosa, Trolox and 17beta-estradiol protect against amyloid beta-peptide neurotoxicity by a mechanism that involves modulation of the Wrt signaling pathway, J Biol Chem, 280 (2005) 11615-11625.
[19] G. V. De Ferrari, M. A. Chacon, M. I. Barria, J. L. Garrido, J. A. Godoy, G. Olivares, A. E. Reyes, A. Alvarez, M. Bronfman, N.C. Inestrosa, Activation of Wnt signaling rescues neurodegeneration and behavioral impairments induced by beta-amyloid fibrils, Mol Psychiatry, 8 (2003) 195-208.
[20] A. R. Alvarez, J A. Godoy, K. Mullendorff, G. H. Olivares, M. Bronfman, N. C. Inestrosa, Wnt-3a overcomes beta-amyloid toxicity in rat hippocampal neurons, Exp Cell Res, 297 (2004) 186-196.
[21] Q. G. Zhang, R. Wang, M. Khan, V. Mahesh, D. W. Brann, Role of Dickkopf-1, an antagonist of the Wnt/beta-catenin signaling pathway, in estrogen-induced neuroprotection and attenuation of tau phosphorylation, J Neurosci, 28 (2008) 8430-8441.
[22] D. C. Lie, S. A. Colamarino, H. J. Song, L. Desire, H. Mira, A. Consiglio, E. S. Lein, S. Jessberger, H. Lansford, A. R. Dearie, F. H. Gage, Wnt signalling regulates adult hippocampal neurogenesis, Nature, 437 (2005) 1370-1375.
[23] T. Kuwabara, J. Hsieh, A. Muotri, G. Yeo, M. Warashina, D. C. Lie, L. Moore, K. Nakashima, M. Asashima, F. H. Gage, Wnt-mediated activation of NeuroD1 and retro-elements during adult neurogenesis, Nat Neurosci, 12 (2009) 1097-1105.
[24] O. Karalay, K. Doberauer, K. C. Vadodaria, M. Knobloch, L. Berti, A. Miquelajauregui, M. Schwark, R. Jagasia, M. M. Taketo, V. Tarabykin, D. C. Lie, S. Jessberger, Prospero-related homeobox 1 gene (Prox1) is regulated by canonical Wnt signaling and has a stage-specific role in adult hippocampal neurogenesis, Proc Natl Acad Sci USA, 108 (2011) 5807-5812.
[25] R. Baron, M. Kneissel, WNT signaling in bone homeostasis and disease: from human mutations to treatments, Nat Med, 19 (2013) 179-192.
[26] T. A. Burgers, B. O. Williams, Regulation of Wnt/beta-catenin signaling within and from osteocytes, Bone, 54 (2013) 244-249.
[27] S. M. Grundy, What is the contribution of obesity to the metabolic syndrome?, Endocrinol Metab Clin North Am, 33 (2004) 267-282, table of contents.
[28] A. H. Mokdad, E. S. Ford, B. A. Bowman, W. H. Dietz, F. Vinicor, V. S. Bales, J. S. Marks, Prevalence of obesity, diabetes, and obesity-related health risk factors, 2001, JAMA, 289 (2003) 76-79.
[29] C. Christodoulides, C. Lagathu, J. K. Sethi, A. Vidal-Puig, Adipogenesis and WNT signalling, Trends Endocrinol Metab, 20 (2009) 16-24.
[30] M. Romero, A. Sabate-Perez, V. A. Francis, I. Castrillon-Rodriguez, A. Diaz-Ramos, M. Sanchez-Feutrie, X. Duran, M. Palacin, J. M. Moreno-Navarrete, B. Gustafson, A. Hammarstedt, J. M. Femandez-Real, J. Vendrell, U. Smith, A. Zorzano, TPS31NP2 regulates adiposity by activating beta-catenin through autophagy-dependent sequestration of GSK3beta, Nat Cell Biol, 20 (2018) 443-454.
[31] T. Jin, The WNT signalling pathway and diabetes mellitus, Diabetologia, 51 (2008) 1771-1780.
[32] C. Lin, W. Lu, L. Zhai, T. Bethea, K. Berry, Z. Qu, W. R. Waud, Y. Li, Mesd is a general inhibitor of different Wnt ligands in Wnt/LRP signaling and inhibits PC-3 tumor growth in vivo, FEBS letters, 585 (2011) 3120-3125.
[33] Y. Li, W. Lu, X. He, A. L. Schwartz, G. Bu, LRP6 expression promotes cancer cell proliferation and tumorigenesis by altering beta-catenin subcellular distribution, Oncogene, 23 (2004) 9129-9135.
[34] J. R. Hill, In Vitro Drug Metabolism Using Liver Microsomes, Current Protocols in Pharmacology, 23 (2003) 7.8.1-7.8.11.
[35] W. Lu, C. Lin, M. J. Roberts, W. R. Waud, G. A. Piazza, Y. Li, Niclosamide suppresses cancer cell growth by inducing Wnt co-receptor LRP6 degradation and inhibiting the Wnt/beta-catenin pathway, PLoS One, 6 (2011) e29290.
[36] K. M. Hennig, D. M. Fass, W. N. Zhao, S. D. Sheridan, T. Fu, S. Erdin, A. Stortchevoi, D. Lucente, J. D. Cody, D. Sweetser, J. F. Gusella, M. E. Talkowski, S. J. Haggarty, WNT/beta-Catenin Pathway and Epigenetic Mechanisms Regulate the Pitt-Hopkins Syndrome and Schizophrenia Risk Gene TCF4, Mol Neuropsychiatry, 3 (2017) 53-71.
[37] S. Prakash, B. J. Foster, M. Meyer, A. Wozniak, L. K. Heilbrun, L. Flaherty, M. Zalupski, L. Radulovic, M. Valdivieso, P. M. LoRusso, Chronic oral administration of CI-994: a phase 1 study, Invest New Drugs, 19 (2001) 1-11.
[38] B. De Strooper, R. Vassar, T. Golde, The secretases: enzymes with therapeutic potential in Alzheimer disease, Nat Rev Neurol, 6 (2010) 99-107.
[39] R. Yan, R. Vassar, Targeting the beta secretase BACE1 for Alzheimer [symbol] disease therapy, Lancet Neurol, 13 (2014) 319-329.
[40] S. Frame, P. Cohen, GSK3 takes centre stage more than 20 years after its discovery, Biochem J, 359 (2001) 1-16.
[41] L. M. Ittner, J. Gotz, Amyloid-beta and tau-a toxic pas de deux in Alzheimer® disease, Nat Rev Neurosci, 12 (2011) 65-72.
[42] G. S. Bloom, Amyloid-beta and tau: the trigger and bullet in Alzheimer disease pathogenesis, JAMA Neurol, 71 (2014) 505-508.
[43] F. Hemandez, E. Gomez de Barreda, A. Fuster-Matanzo, J. J. Lucas, J. Avila, GSK3: a possible link between beta amyloid peptide and tau protein, Exp Neurol, 223 (2010) 322-325.
[44] C. Li, J. Gotz, Tau-based therapies in neurodegeneration: opportunities and challenges, Nat Rev Drug Discov, 16 (2017) 863-883.
[45] E. Tian, F. Zhan, R. Walker, E. Rasmussen, Y. Ma, B. Barlogie, J. D. Shaughnessy, Jr., The role of the Wnt-signaling antagonist DKK1 in the development of osteolytic lesions in multiple myeloma, N Engl J Med, 349 (2003) 2483-2494.
[46] W. Lu, K. A. Kim, J. Liu, A. Abo, X. Feng, X. Cao, Y. Li, R-spondin1 synergizes with Wnt3A in inducing osteoblast differentiation and osteoprotegerin expression, FEBS Lett, 582 (2008) 643-650.
[47] A. G. Kazantsev, L. M. Thompson, Therapeutic application of histone deacetylase inhibitors for central nervous system disorders, Nat Rev Drug Discov, 7 (2008) 854-868.
[48] A. Didonna, P. Opal, The promise and perils of HDAC inhibitors in neurodegeneration, Ann Clin Transl Neurol, 2 (2015) 79-101.
[49] W. A. Banks, From blood-brain barrier to blood-brain interface: new opportunities for CNS drug delivery, Nat Rev Drug Discov, 15 (2016) 275-292.

What is claimed:

1. A compound, or pharmaceutically acceptable salt thereof, having a structure of Formula (I) or (I′): 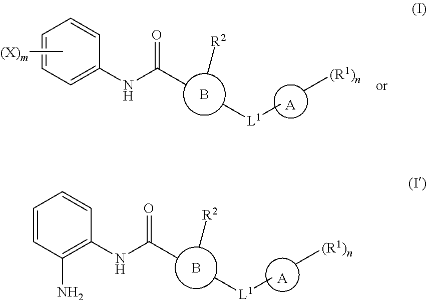
wherein
ring B-R² is 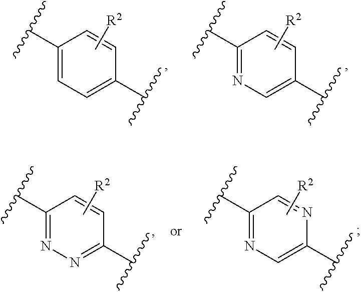
L¹ is NH—CO—C₀₋₃alkylene or CO—NH—C₀₋₄alkylene;
ring A is a 4-12-membered monocyclic, bicyclic, bridged, or spiro heterocycle comprising a nitrogen ring atom;
each R¹ is independently H, C₁₋₆alkyl, halo, C₁₋₆haloalkyl, C₁₋₃alkylene-O—C₁₋₃alkyl, C₀₋₃alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl;
R² is H, F, OH, OMe, or NH₂;
each X is independently NH₂, NMe₂, F, or CF₃;
m is 1 or 2; and
n is 1, 2, or 3,
with the proviso that when ring A comprises piperidinyl, at least one R¹ is other than H.

2. The compound or salt of claim 1, wherein ring B is

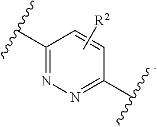

3. The compound or salt of claim 1, wherein ring B is

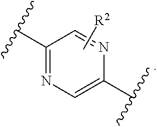

4. The compound or salt of claim 1, wherein ring B is

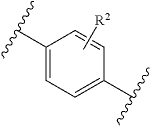

5. The compound or salt of claim 1, wherein ring B is

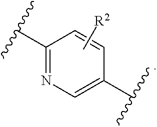

6. The compound or salt of any one of claims 1 to 6, wherein m is 1.

7. The compound or salt of claim 6, wherein X is NMe₂, F, or CF₃.

8. The compound or salt of claim 6, wherein X is NH₂.

9. The compound or salt of any one of claims 1 to 5, wherein m is 2.

10. The compound or salt of claim 9, wherein one X is NH₂ and one X is F.

11. The compound or salt of claim 1, having a structure of Formula (IA), (IA′), (IA″), (IA″′), (IB), (IB′), (IC), or (IC′):

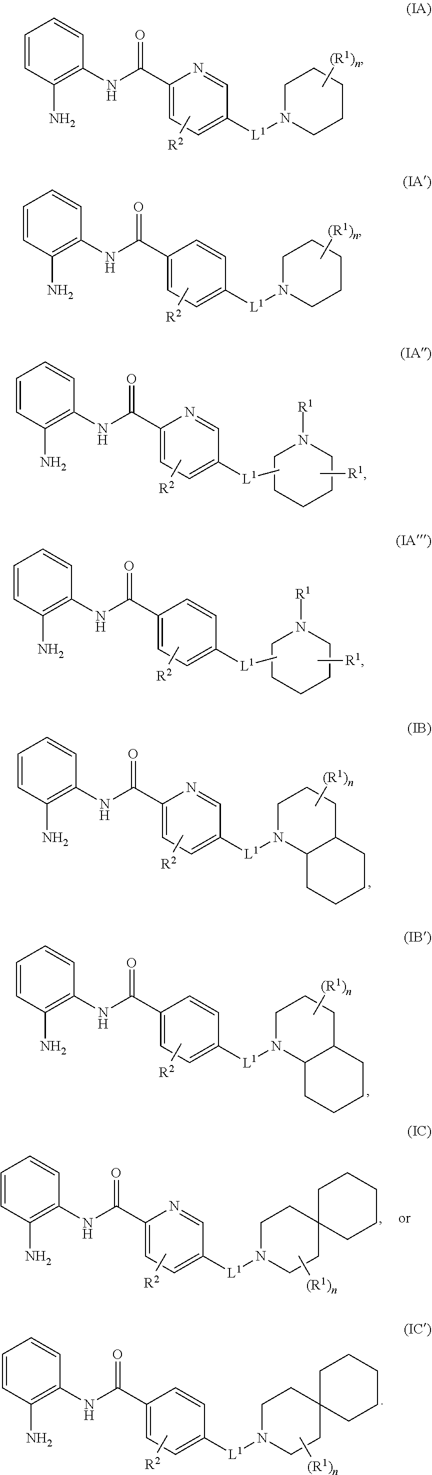

12. The compound or salt of claim 1, having a structure of Formula (ID) or (ID′): 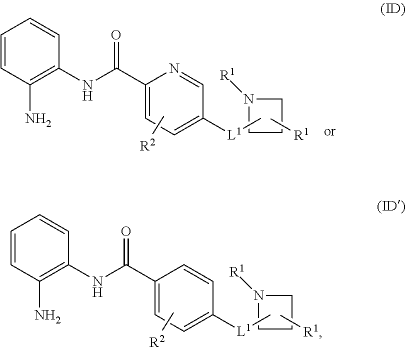
wherein when L¹ is attached to the ring nitrogen, R¹ on the ring nitrogen is null.

13. The compound or salt of claim 1, having a structure of Formula (IE) or (IE′): 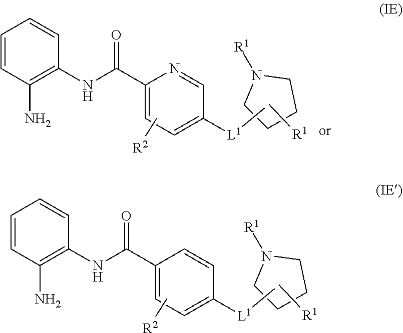
wherein when L¹ is attached to the ring nitrogen, R¹ on the ring nitrogen is null.

14. The compound or salt of any one of claims 1 to 10, wherein ring A comprises azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl.

15. The compound or salt of any one of claims 1 to 14, wherein at least one R¹ is fluoro, cyclopropyl, cyclopentyl, cyclohexyl, cycloheptyl, CH₂cyclohexyl, CH₂cyclopenyl, methyl, CH₂CH₂OCH₃, isopropyl, difluoroethyl, trifluoroethyl, tetrahydropyranyl, CH₂-(methyl-isoxazolyl), methyl-pyrazolyl, CH₂CH₂phenyl, CH₂phenyl, CH₂(methoxyphenyl), or phenyl.

16. The compound or salt of any one of claims 1 to 15, wherein L¹ is NHCO—C₀₋₄alkylene.

17. The compound or salt of any one of claims 1 to 15, wherein L¹ is CONH—C₀₋₄alkylene.

18. The compound or salt of claim 17, wherein L¹ is CONHCH₂.

19. The compound or salt of claim 17, wherein L¹ is CONHCH₂CH₂.

20. The compound or salt of claim 17, wherein L¹ is CONHCH₂CH(CH₃).

21. The compound or salt of any one of claims 1 to 20, wherein R² is F.

22. The compound or salt of any one of claims 1 to 20, wherein R² is H.

23. The compound or slat of any one of claims 1 to 20, wherein R² is NH₂, OMe, or OH.

24. A compound, or pharmaceutically acceptable salt thereof, as listed in Table A.

25. A compound, or pharmaceutically acceptable salt thereof, as listed in Table B.

26. A pharmaceutical composition comprising the compound or salt of any one of claims 1 to 25 and a pharmaceutically acceptable carrier.

27. A method of activating Wnt activity in a cell comprising contacting the cell with an effective amount of the compound or salt of any one of claims 1 to 25.

28. A method of treating a neurological disorder in a subject in need thereof, comprising administering to the subject a therapeutically effective amount of the compound or salt of any one of claims 1 to 25.

29. The method of claim 28, wherein the neurological disorder is selected from the group consisting of Alzheimers disease, frontotemporal dementias, dementia with lewy bodies, a prion disease, Parkinsons disease, Huntingtons disease, progressive supranuclear palsy, corticobasal degeneration, multiple system atrophy, amyotrophic lateral sclerosis (ALS), inclusion body myositis, autism, degenerative myopathy, diabetic neuropathy, endocrine neuropathy, orthostatic hypotension, multiple sclerosis and Charcot-Marie-Tooth disease.

30. The method of claim 28, wherein the neurological disorder is Alzheimer's disease.

31. A method of treating bone degeneration in a subject in need thereof, comprising administering to the subject a therapeutically effective amount of the compound or salt of any one of claims 1 to 25.
